(19) United States
(12) Patent Application Publication
(10) Pub. No.: US 2017/0340307 A1
(43) Pub. Date: Nov. 30, 2017

(54) ULTRASONIC MEASUREMENT APPARATUS

(71) Applicant: SEIKO EPSON CORPORATION, Tokyo (JP)

(72) Inventor: Kazuyuki KANO, Suwa-shi (JP)

(73) Assignee: SEIKO EPSON CORPORATION, Tokyo (JP)

(21) Appl. No.: 15/602,413

(22) Filed: May 23, 2017

(30) Foreign Application Priority Data
May 26, 2016 (JP) 2016-104917

(51) Int. Cl. A61B 8/08; A61B 8/00; A61B 8/14

(52) U.S. Cl. CPC A61B 8/0841; A61B 8/5207; A61B 8/14; A61B 8/4444

(57) ABSTRACT

An ultrasonic measurement apparatus includes an ultrasonic probe that emits an ultrasonic wave to the arm, detects a reflected wave from the arm, and outputs an ultrasonic signal, an image processing unit that forms an ultrasonic image on the basis of the ultrasonic signal, and a first display device and a second display device that displays the ultrasonic image, in which the ultrasonic image includes a blood vessel image indicating a blood vessel included in the arm, and a subtractive color needle image indicating a puncture needle inserted into the blood vessel, and in which predetermined colors corresponding to relative positions between the blood vessel and the puncture needle are displayed in the ultrasonic image.

### BACKGROUND

### 1. Technical Field

The present invention relates to an ultrasonic measurement apparatus.

### 2. Related Art

An ultrasonic measurement apparatus is widely used in which a subject is irradiated with an ultrasonic wave, and an ultrasonic image is displayed by using a reflected wave which is reflected from the inside of the subject. A puncture needle may be inserted into an organ such as a blood vessel located inside a subject by using the ultrasonic measurement apparatus. An ultrasonic measurement apparatus including a guide portion guiding a puncture needle is disclosed in JP-A-2003-334191. In the ultrasonic measurement apparatus, a hole for guiding the puncture needle is provided in the guide portion.

When the puncture needle is inserted into a subject, an operator operates the puncture needle so that the puncture needle enters an ultrasonic wave irradiation range. Consequently, a view of the puncture needle is displayed in an ultrasonic image. The operator checks whether or not the puncture needle correctly advances toward a target location while viewing the ultrasonic image. The operator causes the puncture needle to advance toward the target location by adjusting an angle at which the puncture needle is inserted while viewing the ultrasonic image.

When the puncture needle is inserted while viewing the ultrasonic image, it is necessary to check relative positions between an insertion target object and the puncture needle. The operator inserts the puncture needle so that a tip of the puncture needle is located at the center of the target object. The ultrasonic image in JP-A-2003-334191 is a monochromatic image, and thus relative positions between an appearance of the target object and the tip of the puncture needle are hardly checked. Thus, there is a probability that the puncture needle may break through the target object. Therefore, an ultrasonic measurement apparatus is desired in which an operator can easily recognize a position of a puncture needle relative to a target object when viewing an ultrasonic image.

### SUMMARY

An advantage of some aspects of the invention is to solve the problem described above and the invention can be implemented as the following forms or application examples.

### Application Example 1

An ultrasonic measurement apparatus according to this application example includes an ultrasonic probe that emits an ultrasonic wave to a subject, detects a reflected wave from the subject, and outputs an ultrasonic signal; an image processing unit that forms an ultrasonic image on the basis of the ultrasonic signal; and a display unit that displays the ultrasonic image, in which the ultrasonic image includes a target object image indicating a target object included in the subject, and a needle image indicating a puncture needle inserted into the target object, and in which predetermined colors corresponding to relative positions between the target object and the puncture needle are displayed in the ultrasonic image.

According to this application example, the ultrasonic measurement apparatus includes the ultrasonic probe, the image processing unit, and the display unit. The ultrasonic probe emits an ultrasonic wave to a subject, detects a reflected wave from the subject, and outputs an ultrasonic signal. The image processing unit forms an ultrasonic image on the basis of the ultrasonic signal, and the display unit displays the ultrasonic image. The ultrasonic image includes a target object image and a needle image. A target object into which a puncture needle is inserted is present inside the subject. The target object includes a blood vessel or a nerve fascicle. An operator inserts the puncture needle into the target object. The target object image is an image indicating the target object. The needle image is an image indicating the puncture needle inserted into the subject. Predetermined colors corresponding to relative positions between the target object and the puncture needle are displayed in the ultrasonic image. As a result, the operator can easily recognize a position of the puncture needle relative to the target object by viewing the ultrasonic image.

### Application Example 2

In the ultrasonic measurement apparatus according to the application example, a location where the predetermined colors corresponding to relative positions between the target object and the puncture needle are displayed in the ultrasonic image is the needle image.

According to this application example, the predetermined colors corresponding to relative positions between the target object and the puncture needle are displayed in the needle image. Therefore, an operator can easily recognize relative positions between the target object and the puncture needle by using shapes of the target object and the needle image and color information of the needle image by viewing the needle image. Since the operator has only to look at the needle image, the operator can more easily recognize a position of the puncture needle relative to the target object than in a case where the operator looks at a plurality of locations.

### Application Example 3

In the ultrasonic measurement apparatus according to the application example, the target object has a bar shape, and colors of the needle image are different from each other at a location where the puncture needle has not passed through the center of the target object and a location where the puncture needle has passed through the center of the target object.

According to this application example, the target object has a bar shape. Colors of the needle image are different from each other at a location where the puncture needle has not passed through the center of the target object and a location where the puncture needle has passed through the center of the target object. Therefore, an operator can easily judge whether or not a tip of the puncture needle passes through the center of the target object by viewing the colors of the ultrasonic image of the puncture needle.

### Application Example 4

In the ultrasonic measurement apparatus according to the application example, colors of the needle image are different from each other at a location where the puncture needle is not inserted into the target object and a location where the puncture needle is inserted into the target object.

According to this application example, colors of the needle image are different from each other at a location where the puncture needle is not inserted into the target object and a location where the puncture needle is inserted into the target object. Therefore, an operator can easily recognize a location where a tip of the puncture needle is inserted into the target object by viewing the colors of the ultrasonic image.

### Application Example 5

In the ultrasonic measurement apparatus according to the application example, the target object image is an image obtained by cutting the target object at a plane intersecting an emission direction of the ultrasonic wave along an axis of the target object, and the target object image includes an axis image indicating the axis of the target object.

According to this application example, the target object image is an image obtained by cutting the target object at a plane intersecting an emission direction of the ultrasonic wave along an axis of the target object. The target object image includes an axis image indicating the axis of the target object. When an operator inserts the puncture needle into the target object, the operator inserts the puncture needle along the axis of the target object. Since the target object image includes the axis image, the operator can insert the puncture needle into the target object by using the axis image as a guide.

Therefore, the operator can easily insert the puncture needle along the axis of the target object.

### Application Example 6

In the ultrasonic measurement apparatus according to the application example, the needle image is an image in which the puncture needle is viewed from an emission direction of the ultrasonic wave.

According to this application example, the needle image is an image in which the puncture needle is viewed from the emission direction of the ultrasonic wave. Ina case where the puncture needle is taken as a predetermined section, only a part of the puncture needle is displayed as an image, and thus a position of the puncture needle is hardly recognized. On the other hand, the needle image in the application example is an image in which the puncture needle is viewed from the emission direction of the ultrasonic wave, and thus the needle image shows the entire shape of the puncture needle. Therefore, an operator can easily recognize relative positions between the target object and the puncture needle.

### Application Example 7

In the ultrasonic measurement apparatus according to the application example, a color of the puncture needle has brightness or saturation higher than brightness or saturation of a color of the inside of the target object.

According to this application example, a color of the puncture needle has brightness or saturation higher than brightness or saturation of a color of the inside of the target object. Therefore, since an operator can check the needle image in a bright color on the background in a dark color, the operator can check the needle image on an easily viewable screen.

### Application Example 8

In the ultrasonic measurement apparatus according to the application example, the ultrasonic image includes a mark image indicating a direction in which a front end of the needle image comes close to an axis of the target object image.

According to this application example, the ultrasonic image includes a mark image indicating a direction in which a front end of the needle image indicating the puncture needle comes close to an axis of the target object image indicating the target object. Therefore, an operator can easily cause the tip of the puncture needle to come close to the axis of the target object by viewing the mark image.

### Application Example 9

In the ultrasonic measurement apparatus according to the application example, the display unit is provided in the ultrasonic probe.

According to this application example, the display unit is provided in the ultrasonic probe, and since an operator inserts the puncture needle from a location close to the ultrasonic probe, the hands of the operator are located at the location close to the ultrasonic probe. Therefore, since the display unit and the hands of the operator can be made to come close to each other, the operator can check the display unit and the hands through short visual line movement. As a result, the operator can operate the puncture needle while viewing the display unit, and can thus easily perform an operation of causing the tip of the puncture needle to come close to the target object.

### Application Example 10

In the ultrasonic measurement apparatus according to the application example, the target object image includes an image obtained by cutting the target object at a plane intersecting the axis of the target object and passing in the emission direction of the ultrasonic wave, and the needle image includes an image in which a tip of the puncture needle is viewed from an axial direction of the target object.

According to this application example, the target object image includes an image obtained by cutting the target object at a plane intersecting the axis of the target object and passing in the emission direction of the ultrasonic wave. The needle image includes an image in which a tip of the puncture needle is viewed from an axial direction of the target object. The target object image includes a view in which the target object and the tip of the puncture needle are viewed from the axial direction. Therefore, an operator can check relative positions between the target object and the tip of puncture needle viewed from the axial direction. As a result, the operator can more easily recognize a position of the puncture needle relative to the target object.

### Application Example 11

In the ultrasonic measurement apparatus according to the application example, the target object image includes an image obtained by cutting the target object at a plane passing in the emission direction of the ultrasonic wave along the axis of the target object, and the needle image includes an image in which the puncture needle is viewed from a direction intersecting an axial direction of the target object and intersecting the emission direction of the ultrasonic wave.

According to this application example, the target object image includes an image obtained by cutting the target object at a plane passing in the emission direction of the ultrasonic wave along the axis of the target object. The needle image includes an image in which the puncture needle is viewed from a direction intersecting an axial direction of the target object and intersecting the emission direction of the ultrasonic wave. The ultrasonic image includes a view in which the target object and the puncture needle are viewed from a direction intersecting the axial direction and the emission direction of the ultrasonic wave. Therefore, an operator can check relative positions between the target object and the puncture needle viewed from the direction intersecting the axial direction and the emission direction of the ultrasonic wave. As a result, the operator can more easily recognize a position of the puncture needle relative to the target object.

### BRIEF DESCRIPTION OF THE DRAWINGS

The invention will be described with reference to the accompanying drawings, wherein like numbers reference like elements.

FIG. 1 is a schematic perspective view illustrating a configuration of an ultrasonic measurement apparatus.

FIG. 2 is a schematic side sectional view illustrating a structure of an ultrasonic probe.

FIG. 3 is a schematic side sectional view illustrating a structure of the ultrasonic probe.

FIG. 4 is an electrical control block diagram of the ultrasonic measurement apparatus.

FIG. 5 is a flowchart illustrating a puncture needle insertion method.

FIG. 6 is a schematic diagram for explaining the puncture needle insertion method.

FIG. 7 is a schematic diagram for explaining the puncture needle insertion method.

FIG. 8 is a schematic diagram for explaining the puncture needle insertion method.

FIG. 9 is a schematic diagram for explaining the puncture needle insertion method.

FIG. 10 is a schematic diagram for explaining the puncture needle insertion method.

FIG. 11 is a schematic diagram for explaining the puncture needle insertion method.

FIG. 12 is a schematic diagram for explaining the puncture needle insertion method.

FIG. 13 is a schematic diagram for explaining the puncture needle insertion method.

FIG. 14 is a schematic diagram for explaining the puncture needle insertion method.

FIG. 15 is a schematic diagram for explaining the puncture needle insertion method.

FIG. 16 is a schematic diagram for explaining the puncture needle insertion method.

FIG. 17 is a schematic diagram for explaining the puncture needle insertion method.

FIG. 18 is a schematic diagram for explaining the puncture needle insertion method.

FIG. 19 is a schematic diagram for explaining the puncture needle insertion method.

FIG. 20 is a schematic diagram for explaining the puncture needle insertion method.

FIG. 21 is a schematic diagram for explaining the puncture needle insertion method.

FIG. 22 is a schematic diagram for explaining the puncture needle insertion method.

FIG. 23 is a schematic diagram for explaining the puncture needle insertion method.

FIG. 24 is a schematic diagram for explaining the puncture needle insertion method.

FIG. 25 is a schematic diagram for explaining the puncture needle insertion method.

FIG. 26 is a schematic diagram for explaining the puncture needle insertion method.

FIG. 27 is a schematic diagram for explaining the puncture needle insertion method.

FIG. 28 is a schematic diagram for explaining the puncture needle insertion method.

FIG. 29 is a schematic diagram for explaining the puncture needle insertion method.

FIG. 30 is a schematic diagram for explaining the puncture needle insertion method.

FIG. 31 is a schematic diagram for explaining the puncture needle insertion method.

FIG. 32 is a schematic diagram for explaining the puncture needle insertion method.

FIG. 33 is a schematic diagram for explaining the puncture needle insertion method.

FIG. 34 is a schematic diagram for explaining the puncture needle insertion method.

FIG. 35 is a schematic diagram for explaining the puncture needle insertion method.

FIG. 36 is a schematic diagram for explaining the puncture needle insertion method.

FIG. 37 is a schematic diagram for explaining the puncture needle insertion method.

### DESCRIPTION OF EXEMPLARY EMBODIMENTS

Hereinafter, an embodiment will be described with reference to the drawings. Respective members in the drawings are illustrated in different scales so that the respective members have recognizable sizes on the drawings.

### Embodiment

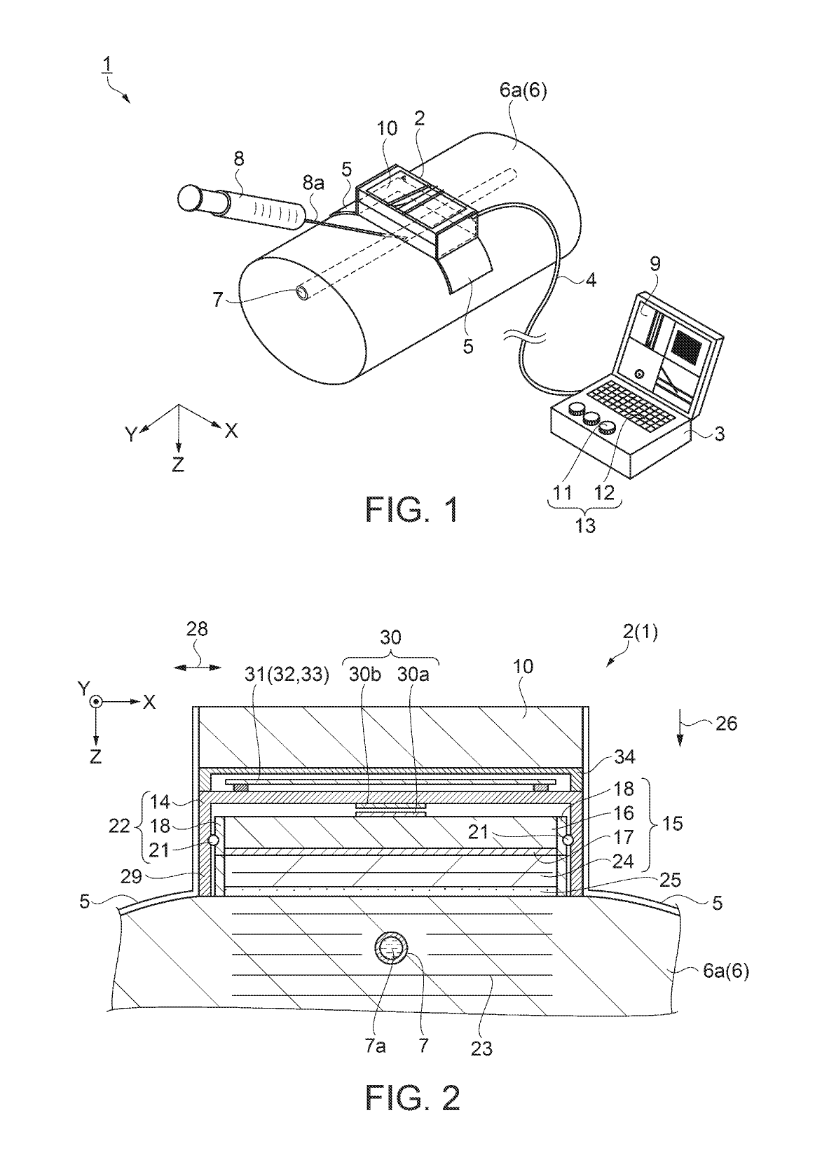

In the present embodiment, with reference to the drawings, a description will be made of characteristic examples of an ultrasonic measurement apparatus and a method of inserting a puncture needle into a blood vessel by using the ultrasonic measurement apparatus. The ultrasonic measurement apparatus according to the embodiment will be described with reference to FIGS. 1 to 4. FIG. 1 is a schematic perspective view illustrating a configuration of the ultrasonic measurement apparatus. As illustrated in FIG. 1, an ultrasonic measurement apparatus 1 includes an ultrasonic probe 2 and a control device 3, and the ultrasonic probe 2 and the control device 3 are connected to each other via a wiring 4.

The ultrasonic probe 2 is fixed to an arm 6a of a human body 6 as a subject with a tape 5. A blood vessel 7 as a target object is present inside the arm 6a along the arm 6a. A direction in which the blood vessel 7 extends along the arm 6a is set to a Y direction, and a direction directed toward the blood vessel 7 from a surface of the arm 6a is set a Z direction. A direction which is orthogonal to the Y direction the Z direction is set to an X direction. The blood vessel 7 is one of parts extending in a bar shape inside the human body 6.

An operator inserts a puncture needle 8a of a syringe 8 into the arm 6a toward the blood vessel 7. The blood vessel 7 maybe a vein, and may be an artery. When the operator inserts the puncture needle 8a into the blood vessel 7 and a tip of the puncture needle 8a reaches the inside of the blood vessel 7, the operator stop movement of the puncture needle 8a. In this state, the operator injects a liquid medicine into the blood vessel 7. Alternatively, the operator sucks blood in the blood vessel 7 into the syringe 8.

The ultrasonic probe 2 emits ultrasonic waves toward the puncture needle 8a and the blood vessel 7 in the arm 6a. Reflected waves which are reflected from the puncture needle 8a and the blood vessel 7 are received so as to be converted into electric signals. The electric signals are converted into digital signals and are then transmitted to the control device 3 via the wiring 4. The digital signals correspond to ultrasonic signals. The control device 3 is provided with a first display device 9 as a display unit, and an ultrasonic image formed on the basis of the digital signals is displayed on the first display device 9. The ultrasonic image represents a video of the inside of the arm 6a detected by using the reflected waves of the ultrasonic waves. The ultrasonic probe 2 is provided with a second display device 10 as a display unit. The ultrasonic image is also displayed on the second display device 10.

The control device 3 is provided with an input device 13 such as a rotary knob 11 and a keyboard 12. The operator operates the input device 13 so as to adjust a traveling direction or the intensity of an ultrasonic wave emitted from the ultrasonic probe 2. The operator checks a position of the tip of the puncture needle 8a relative to the blood vessel 7 while viewing the first display device 9 and the second display device 10, and inserts the puncture needle 8a toward the blood vessel 7. When the tip of the puncture needle 8a enters the blood vessel 7, the operator stops movement of the puncture needle 8a. Next, the operator injects a liquid medicine or collects blood.

Two persons preferably perform an operation, but a single person may perform an operation. When a single person performs an operation, the operator checks relative positions between the blood vessel 7 and the puncture needle 8a while viewing the second display device 10. Since the operator inserts the puncture needle 8a from a location close to the ultrasonic probe 2, the hands of the operator are located at the location close to the ultrasonic probe 2. Therefore, since the second display device 10 and the hands of the operator come close to each other, the operator can check the second display device 10 and the hands through short visual line movement. As a result, the operator can operate the puncture needle 8a while viewing the second display device 10, and can thus easily perform an operation of causing the tip of the puncture needle 8a to come close to an axis of the blood vessel 7.

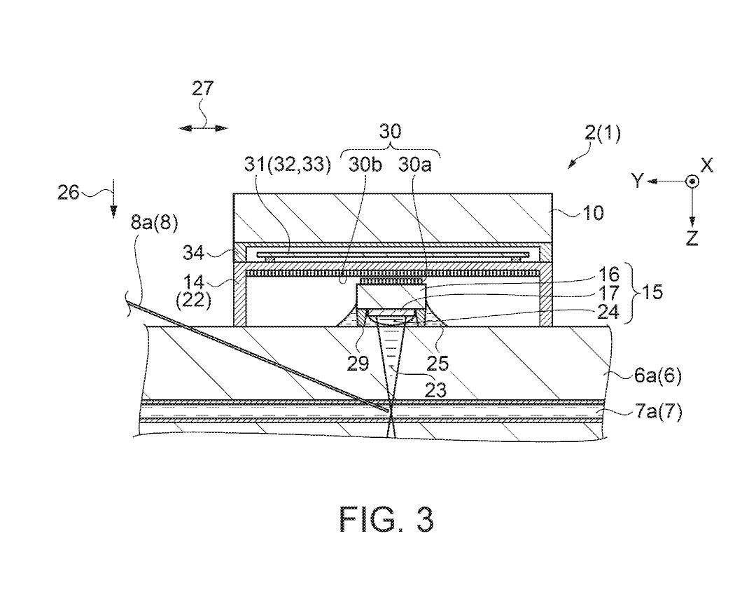

FIGS. 2 and 3 are schematic sectional views illustrating a structure of the ultrasonic probe. FIG. 2 is a view which is viewed from a longitudinal direction of the blood vessel 7, and FIG. 3 is a view which is viewed from a direction orthogonal to the longitudinal direction of the blood vessel 7. As illustrated in FIGS. 2 and 3, the blood vessel 7 is located inside the arm 6a, and blood 7a flows in the blood vessel 7.

The ultrasonic probe 2 is provided with a first support member 14 having a bottomed rectangular cylindrical shape. A moving object 15 which moves in the Y direction is provided in the first support member 14. The moving object 15 includes a substrate 16, and is provided with an ultrasonic element array 17 on a surface of the substrate 16 on the +Z direction side.

Second support members 18 are provided on both surfaces of the substrate 16 on the ±X direction sides, and the substrate 16 is supported in a state of being interposed between the second support members 18. The second support member 18 on the +X direction side has a groove extending in the Y direction on the surface on the +X direction side. The first support member 14 has a groove extending in the Y direction on an inner surface thereof on the −X direction side. A plurality of balls 21 are provided between the respective grooves of the first support member 14 and the second support members 18. The moving object 15 and the first support member 14 on the −X direction side have the same structures as those of the moving object 15 and the first support member 14 on the +X direction side. A linear guide 22 is formed of the first support member 14, the second support members 18, and the balls 21. The balls 21 roll in the linear guide 22, and thus frictional resistance during movement is reduced.

Vibration plates are provided in a matrix on a silicon substrate in the ultrasonic element array 17. A piezoelectric element is provided on each vibration plate. An AC waveform is applied to the piezoelectric element. Consequently, the piezoelectric element causes the vibration plate to vibrate, and thus an ultrasonic wave 23 is emitted. An ultrasonic element is mainly formed of the vibration plate and the piezoelectric element. The emitted ultrasonic wave 23 travels through the arm 6a, and is reflected at the blood vessel 7 or the puncture needle 8a. The ultrasonic element array 17 receives a reflected wave of the ultrasonic wave 23. Apart of the reflected ultrasonic wave 23 causes the vibration plate to vibrate, and thus enlarges and contracts the piezoelectric element. Consequently, the piezoelectric element outputs a voltage signal corresponding to the reflected wave. The control device 3 forms an ultrasonic image by using the voltage signal output from each piezoelectric element.

In the ultrasonic element array 17, a single ultrasonic element may perform both of emission and reception of an ultrasonic wave. Ultrasonic elements having favorable ultrasonic wave emission characteristics and ultrasonic elements having favorable ultrasonic wave reception sensitivity may be arranged. The type of piezoelectric element is not particularly limited, but a piezoelectric element such as a lead zirconate titanate (PZT) element or a polyvinylidene fluoride (PDVF) element may be used. In the present embodiment, the PZT element is used as the piezoelectric element.

An acoustic lens 24 is provided on the arm 6a side of the ultrasonic element array 17. A gel 25 is applied on a skin surface, and the gel 25 is disposed between the acoustic lens 24 and the arm 6a. The gel 25 adjusts acoustic impedance between the acoustic lens 24 and the arm 6a. The ultrasonic waves 23 are hardly reflected due to the gel 25 when entering the arm 6a from the acoustic lens 24. Consequently, the ultrasonic probe 2 can emit the ultrasonic waves 23 into the arm 6a with high efficiency.

A direction in which the ultrasonic element array 17 emits the ultrasonic waves 23 is set to an emission direction 26. The emission direction 26 is the Z direction. A direction in which the axis of the blood vessel 7 extends is set to a blood vessel axis direction 27 as an axial direction of a target object. The blood vessel axis direction 27 is the same as a direction in which the moving object 15 moves, and is the Y direction. The acoustic lens 24 has a shape obtained by cutting a cylinder at a plane which is parallel to an axis of the cylinder. A direction in which the axis of the cylinder extends is set to a lens axis direction 28. The lens axis direction 28 is the X direction.

An elastic packing 29 is provided on the surface of the substrate 16 on the +Z direction side. The packing 29 is provided to surround the ultrasonic element array 17. In a case where the moving object 15 moves, the packing 29 slides on the skin of the arm 6a. The gel 25 is disposed between the skin and the acoustic lens 24, and the gel 25 is also surrounded by the packing 29. In a case where the moving object 15 moves, the gel 25 also moves along with the moving object 15. As mentioned above, the gel 25 is normally present between the ultrasonic element array 17 and the acoustic lens 24.

A change in the acoustic impedance between the ultrasonic element array 17 and the acoustic lens 24 can be reduced by using the gel 25. As a result, it is possible to prevent the ultrasonic waves 23 from being reflected between the ultrasonic element array 17 and the acoustic lens 24.

A permanent magnet 30a is provided on the surface of the substrate 16 on the −Z direction side. The permanent magnet 30a is magnetized in which S-poles and N-poles are alternately arranged with fine pitches in the Y direction. An electromagnet 30b is provided on the first support member 14 on the +Z direction side. Coils are disposed to be arranged in the Y direction in the electromagnet 30b. A linear motor 30 as a movement unit is formed of the permanent magnet 30a and the electromagnet 30b. In the linear motor 30, a current flowing through the coils switches, and thus switching occurs between the S-pole and the N-pole. The linear motor 30 causes a Lorentz force to act between the permanent magnet 30a and the electromagnet 30b, and thus the moving object 15 is moved.

A mechanism which linearly moves the moving object 15 is not limited to the electromagnetic linear motor 30. In addition to the linear motor 30, various movement mechanisms such as a linear piezoelectric motor which is driven with piezoelectric elements, and a linear resonance actuator which is moved due to vibration may be used.

A circuit board 31 is provided on the first support member 14 on the −Z direction side. A motor driving circuit 32 driving the linear motor 30 and a transducer driving circuit 33 driving the ultrasonic element array 17 are mounted on the circuit board 31. The circuit board 31 is connected to the control device 3 via the wiring 4. A casing 34 converting the circuit board 31 is provided on the first support member 14 on the −Z direction side. The casing 34 prevents the circuit board 31 from being short-circuited or contaminated. The second display device 10 is provided on the casing 34 on the −Z direction side.

As illustrated in FIG. 2, the ultrasonic element array 17 has a shape which is long in the lens axis direction 28. An ultrasonic image displayed on the first display device 9 is formed by using an image of a plane passing in the lens axis direction 28 and the emission direction 26.

As illustrated in FIG. 3, the linear motor 30 moves the moving object 15 in the blood vessel axis direction 27. In other words, the linear motor 30 moves the acoustic lens 24 and the ultrasonic element array 17 relative to the arm 6a. Consequently, reflected waves can be detected by emitting the ultrasonic waves to the arm 6a in a region in which the ultrasonic element array 17 is moved in the blood vessel axis direction 27. When the puncture needle 8a is located in the emission direction 26 of the ultrasonic element array 17, some of the reflected waves of the ultrasonic waves 23, reflected at the puncture needle 8a and the blood vessel 7, are input to the ultrasonic element array 17.

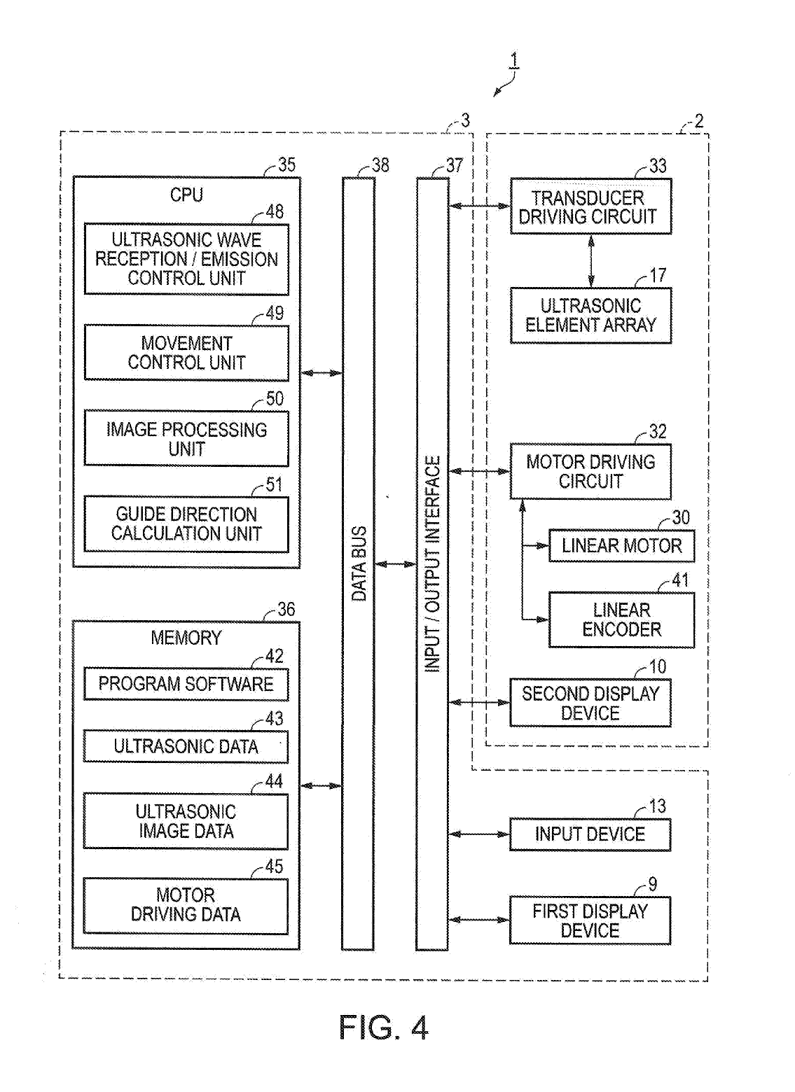

FIG. 4 is an electrical control block diagram of the ultrasonic measurement apparatus. In FIG. 4, the ultrasonic measurement apparatus 1 includes the control device 3 controlling an operation of the ultrasonic measurement apparatus 1. The control device 3 includes a central processing unit (CPU) 35 performing various calculation processes as a processor, and a memory 36 storing various pieces of information. The transducer driving circuit 33, the motor driving circuit 32, the second display device 10, the input device 13, and the first display device 9 are connected to the CPU 35 via an input/output interface 37 and a data bus 38.

The transducer driving circuit 33 is a device driving the ultrasonic element array 17. The transducer driving circuit 33 receives an instruction signal from the CPU 35. The ultrasonic element array 17 is provided with transducers. The transducer driving circuit 33 sequentially causes a transducer at a predetermined location to vibrate. The ultrasonic wave 23 is emitted at the location where the transducer vibrates. The emitted ultrasonic waves 23 are reflected at the blood vessel 7 or the puncture needle 8a, and some of the ultrasonic waves 23 reach the ultrasonic element array 17. In the ultrasonic element array 17, the transducers vibrate due to the received ultrasonic waves 23, and voltage signals are output to the transducer driving circuit 33. The transducer driving circuit 33 receives the voltage signals, and outputs ultrasonic signals obtained by converting the voltage signals into digital signals, to the CPU 35.

The motor driving circuit 32 is a device driving the linear motor 30 and a linear encoder 41. The first support member 14 is provided with the linear encoder 41, and the linear encoder 41 detects a position of the moving object 15. The motor driving circuit 32 receives an instruction signal from the CPU 35. A position and a movement velocity of the moving object 15 are detected by using the linear encoder 41. The motor driving circuit 32 drives the linear motor 30 so that the moving object 15 is located at a position indicated by the instruction signal.

The input device 13 includes not only the rotary knob 11 and the keyboard 12, but also a device performing wired and wireless communication with an external computer. Various pieces of data is input to the CPU 35 and the memory 36 by using the input device 13. The operator operates the input device 13 so as to input measurement conditions.

The first display device 9 and the second display device 10 are display devices such as liquid crystal displays (LCDs) or organic light emitting diodes (OLEDs). The first display device 9 and the second display device 10 display measurement conditions or an ultrasonic image as a measurement result.

The ultrasonic probe 2 is provided with the transducer driving circuit 33, the ultrasonic element array 17, the motor driving circuit 32, the linear motor 30, the second display device 10, and the like. The ultrasonic probe 2 emits the ultrasonic waves 23 to the arm 6a, detects reflected waves from the arm 6a, and outputs ultrasonic signals.

The memory 36 includes semiconductor memories such as a RAM and a ROM, and external storage devices such as a hard disk and a DVD-ROM. In terms of a function, a storage region storing program software 42 in which control procedures for an operation of the ultrasonic measurement apparatus 1 are described, and a storage region storing ultrasonic data 43 detected by the ultrasonic element array 17 are set in the memory 36. A storage region storing ultrasonic image data 44 which is data regarding an ultrasonic image formed by using the ultrasonic data 43 is set. A storage region storing motor driving data 45 which is data regarding conditions for driving the linear motor 30 is set. A storage region which functions as a work area for the CPU 35 or a temporary file, and other various storage regions are set.

The CPU 35 emits the ultrasonic waves 23 to the arm 6a, detects reflected waves, and generates and displays an ultrasonic image, according to the program software 42 stored in the memory 36. The CPU 35 includes an ultrasonic wave reception/emission control unit 48 as a specific function realizing unit. The ultrasonic wave reception/emission control unit 48 performs control of causing the transducer driving circuit 33 to drive the ultrasonic element array 17, and of acquiring data regarding reflected waves of the ultrasonic waves 23.

The CPU 35 includes a movement control unit 49. The movement control unit 49 receives position data of the moving object 15 detected by the linear encoder 41. The movement control unit 49 performs control of moving the moving object 15 at a predetermined speed. The movement control unit 49 acquires the ultrasonic data 43 in an adjusted range in cooperation with the ultrasonic wave reception/emission control unit 48.

The CPU 35 includes an image processing unit 50. The image processing unit 50 receives ultrasonic signals obtained by converting the electric signals based on the reflected waves output from the transducer driving circuit 33, into the digital data. An ultrasonic image is formed by using the ultrasonic signals based on the reflected waves. In other words, the ultrasonic image is formed on the basis of the ultrasonic signals output from the ultrasonic probe 2. The CPU 35 includes a guide direction calculation unit 51. The guide direction calculation unit 51 recognizes the axis of the blood vessel 7 and a tip position of the puncture needle 8a. A direction in which the tip position of the puncture needle 8a comes close to the axis of the blood vessel 7 is calculated. In the present embodiment, the above-described respective functions are realized by using the CPU 35 according to the program software, but, in a case where the above-described respective functions can be realized by independent electronic circuits (hardware) not using the CPU 35, such electronic circuits may be used.

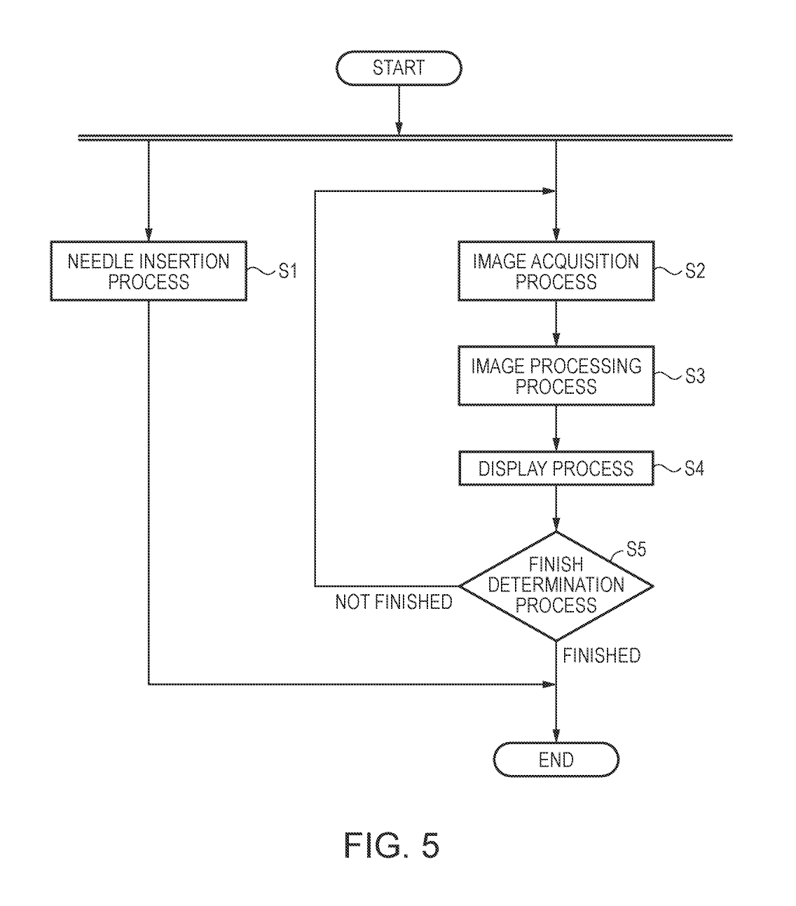

Next, a description will be made of a puncture needle insertion method of inserting the puncture needle 8a up to the blood vessel 7 by using the above-described ultrasonic measurement apparatus 1, with reference to FIGS. 5 to 37. FIG. 5 is a flowchart illustrating a puncture needle insertion method. An operation on the ultrasonic measurement apparatus 1 is performed by two operators such as a first operator and a second operator.

In the flowchart illustrated in FIG. 5, step S1 is executed in applicable to steps S2 to S5. Step S1 corresponds to a needle insertion process. In this process, an operator inserts the puncture needle 8a into the arm 6a. At this time, the operator inserts the puncture needle 8a while viewing ultrasonic images displayed on the first display device 9 and the second display device 10.

Step S2 corresponds to an image acquisition process. In this process, an ultrasonic image is acquired. First, the ultrasonic wave reception/emission control unit 48 causes the transducer driving circuit 33 to drive the ultrasonic element array 17, so as to emit the ultrasonic waves 23 toward the inside of the arm 6a. Some of the ultrasonic waves 23 are reflected at the blood vessel 7 or the puncture needle 8a. Some of the reflected ultrasonic waves 23 reach the ultrasonic element array 17. In the ultrasonic element array 17, the transducers vibrate due to the ultrasonic waves 23 having reached, and voltage signals which are proportional to intensities of the ultrasonic waves 23 are output to the transducer driving circuit 33. The transducer driving circuit 33 stores, in the memory 36, the ultrasonic data 43 including ultrasonic signals obtained by converting the voltage signals which are proportional to the intensities of the ultrasonic waves 23 into digital data. Step S3 corresponds to an image processing process. In this process, the image processing unit 50 receives the ultrasonic data 43 from the memory 36. The image processing unit 50 combines the ultrasonic data 43 with each other, and stores the ultrasonic image data 44 which is data regarding a combined ultrasonic image in the memory 36. Next, the flow proceeds to step S4.

Step S4 corresponds to a display process. In this process, the first display device 9 and the second display device 10 displays the ultrasonic image data 44. Next, the flow proceeds to step S5. Step S5 corresponds to a finish determination process. In this process, it is determined whether or not display of ultrasonic images of the puncture needle 8a is finished. In this step, the operator checks the ultrasonic images displayed on the first display device 9 and the second display device 10. When the tip of the puncture needle 8a does not arrive at the center of the blood vessel 7, the operator judges that the display of the ultrasonic images of the puncture needle 8a is not finished. The flow proceeds to step S2. When the puncture needle 8a reaches the blood vessel 7, and the tip of the puncture needle 8a is inserted into the center of the blood vessel 7, the operator judges that the display of the puncture needle 8a is finished. The process of inserting the puncture needle 8a into the blood vessel 7 is finished. Although not illustrated in the flowchart, a process of injecting a liquid medicine or a process of sucking blood may be performed after the process of inserting the puncture needle 8a into the blood vessel 7.

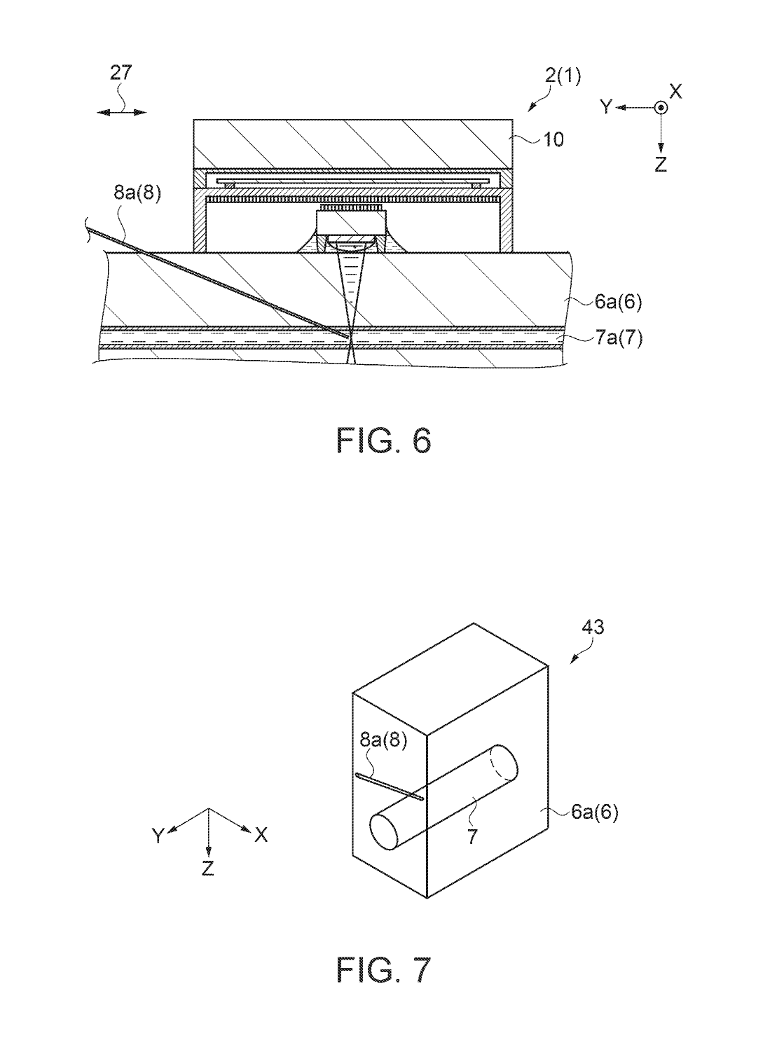

FIGS. 6 to 37 are schematic diagrams for explaining a puncture needle insertion method. Next, with reference to FIGS. 6 to 37, the puncture needle insertion method will be described in detail so as to correspond to steps S1 to S5 illustrated in FIG. 5. FIGS. 6 and 7 are diagrams respective corresponding to the needle insertion process in step S1 and the image acquisition process in step S2. As illustrated in FIG. 6, in step S1, the operator inserts the puncture needle 8a into the arm 6a, and advances the needle tip toward the blood vessel 7. An ultrasonic image is displayed on the second display device 10 of the ultrasonic probe 2. Images indicating the blood vessel 7 and the puncture needle 8a are displayed on the ultrasonic image, and thus the operator inserts the puncture needle 8a while viewing the ultrasonic image.

In step S2, the ultrasonic probe 2 acquires the ultrasonic data 43. The ultrasonic wave reception/emission control unit 48 outputs an instruction signal for emitting the ultrasonic waves 23 to the transducer driving circuit 33. The transducer driving circuit 33 drives the ultrasonic element array 17 in response to the instruction signal. Consequently, the ultrasonic element array 17 emits the ultrasonic waves 23 toward the inside of the arm 6a. Some of the ultrasonic waves 23 are reflected at the blood vessel 7 or the puncture needle 8a. Some of the reflected ultrasonic waves 23 reach the ultrasonic element array 17. In the ultrasonic element array 17, the transducers vibrate due to the ultrasonic waves 23 having reached, and voltage signals which are proportional to intensities of the ultrasonic waves 23 are output to the transducer driving circuit 33. The transducer driving circuit 33 stores, in the memory 36, the ultrasonic data 43 including ultrasonic signals obtained by converting the voltage signals which are proportional to the intensities of the ultrasonic waves 23 into digital data.

The control device 3 moves the moving object 15 in parallel to acquisition of the ultrasonic data 43. A movement aspect of the moving object 15 is not particularly limited, but, in the present embodiment, for example, the moving object 15 moves from an end on the +Y direction side to an end on the −Y direction side in the blood vessel axis direction 27 step by step. When the moving object 15 reaches the end on the −Y direction side, the movement control unit 49 moves the moving object 15 to the end on the +Y direction side at a high speed.

In a case where the ultrasonic data 43 is stored in the memory 36, the movement control unit 49 outputs an instruction signal for driving the linear motor 30 to the motor driving circuit 32. The motor driving circuit 32 drives the linear motor 30 by only one step in response to the instruction signal. When the moving object 15 moves by one step, the ultrasonic wave reception/emission control unit 48 outputs an instruction signal for acquiring an ultrasonic signal to the transducer driving circuit 33. As mentioned above, the process in which the ultrasonic element array 17 and the transducer driving circuit 33 acquires the ultrasonic data 43 and stores the ultrasonic data 43 in the memory 36 and the process in which the moving object 15 moves by one step are alternately performed.

As a result, as illustrated in FIG. 7, the ultrasonic data 43 indicating a structure of the three-dimensional arm 6a including the blood vessel 7 and the puncture needle 8a is stored in the memory 36. The ultrasonic data 43 is an aggregate of data. FIG. 7 is a perspective view illustrating an internal structure of the arm 6a indicated by the three-dimensional ultrasonic data 43. The ultrasonic data 43 indicates reflection intensities of the ultrasonic waves 23 at respective XYZ coordinates. The blood vessel 7 has a tubular shape, and an outer circumferential surface and an inner circumferential surface of the blood vessel 7 can be recognized from the ultrasonic data 43 of the blood vessel 7. An outer circumferential surface and the tip of the puncture needle 8a can be recognized from the ultrasonic data 43 of the puncture needle 8a.

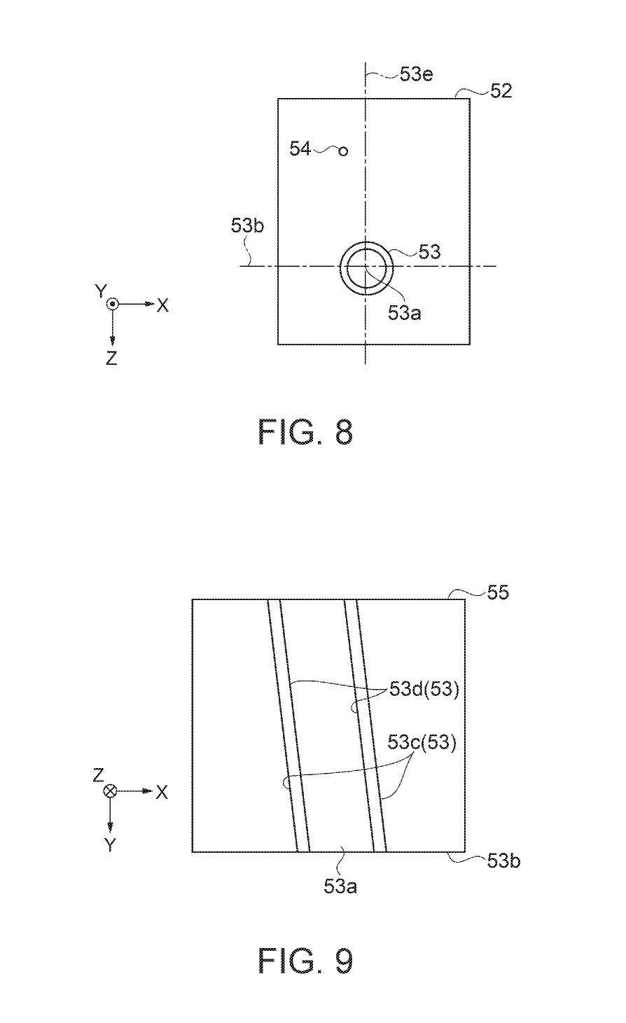

FIGS. 8 to 37 are diagrams corresponding to the image processing process in step S3. As illustrated in FIG. 8, in step S3, the image processing unit 50 forms a first screen 52 at the end on the +Y direction side on the basis of the ultrasonic data 43. The first screen 52 is an image which the blood vessel 7 and the syringe 8 are viewed from the Y direction side, and is referred to as a B mode image. The first screen 52 includes a blood vessel image 53 as a target object image which is an image of the blood vessel 7, and a needle image 54 which is an image of the puncture needle 8a. The image processing unit 50 calculates a coordinate of a center 53a of the blood vessel image 53. Next, a first virtual line 53b which extends through the center 53a in the X direction is set. The image processing unit 50 forms a second screen at the end on the −Y direction side on the basis of the ultrasonic data 43. The second screen includes the blood vessel image 53 which is an image of the blood vessel 7. The image processing unit 50 calculates a coordinate of the center of the blood vessel image 53 on the second screen. Next, a virtual line extending through the center 53a in the second screen in the X direction is set.

The image processing unit 50 provides a first virtual plane directed toward the −Z direction side through the first virtual line 53b in the first screen 52 and the virtual line in the second screen. Next, as illustrated in FIG. 9, the image processing unit 50 calculates a third screen 55 in which the first virtual plane is viewed from the −Z direction side. The blood vessel image 53 is displayed on the third screen 55. An outer wall image 53c indicating an outer wall of the blood vessel 7 is displayed in the blood vessel image 53. An inner wall image 53d indicating an inner wall of the blood vessel 7 is displayed in the blood vessel image 53. The blood vessel image 53 on the third screen 55 is an image obtained by cutting the blood vessel 7 at a plane intersecting the emission direction 26 along the axis of the blood vessel 7.

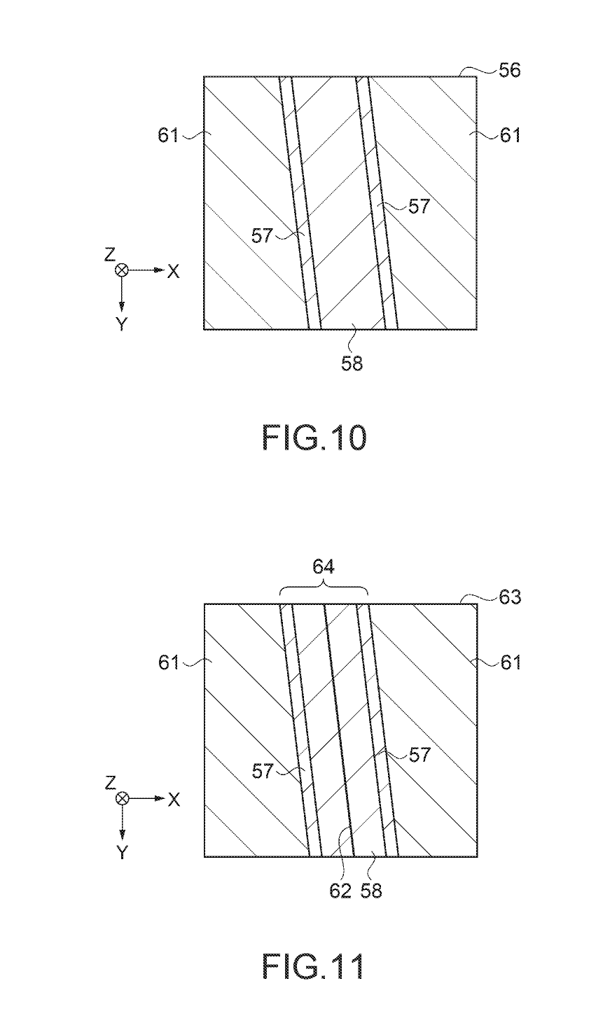

Next, as illustrated in FIG. 10, the image processing unit 50 calculates a fourth screen 56 in which each region is displayed in a predetermined color by performing a subtractive color process such as binarization on the third screen 55. A predetermined color set in each region is not particularly limited. In the present embodiment, for example, a pair of blood vessel wall images 57 interposed between the outer wall image 53c and the inner wall image 53d is displayed white, and a blood vessel inside image 58 interposed between the pair of blood vessel wall images 57 is displayed black. A tissue image 61 indicating a typical tissue located on the right and left in the figure of the blood vessel wall images 57 is displayed gray. Gray is realized by alternately disposing white and black in a matrix. The brightness of gray is adjusted by changing an area ratio between white and black. Therefore, the fourth screen 56 is formed of white pixels and black pixels. In the above-described way, it is possible to reduce a storage capacity required for color data display of a gray portion.

Next, as illustrated in FIG. 11, the image processing unit 50 calculates an axis image 62 indicating the axis of the blood vessel 7, and calculates a fifth screen 63 obtained by combining the fourth screen 56 with the axis image 62. The image processing unit 50 calculates the median line of the pair of blood vessel wall images 57, and uses the median line as the axis image 62. A color of the axis image 62 is not particularly limited, but is set to, for example, blue, in the present embodiment. The blood vessel inside image 58 is black, and the axis image 62 is a blue line with the black background and can thus be clearly recognized. A blood vessel image 64 as a target object image is formed of the blood vessel wall images 57 and the blood vessel inside image 58, and the blood vessel image 64 includes the axis image 62 indicating the axis of the blood vessel 7. The blood vessel image 64 is an image obtained by cutting the blood vessel 7 at a plane intersecting the emission direction 26 along the axis of the blood vessel 7 in the same manner as the blood vessel image 53 on the third screen 55. In step S4, the fifth screen 63 is displayed on the first display device 9 and the second display device 10.

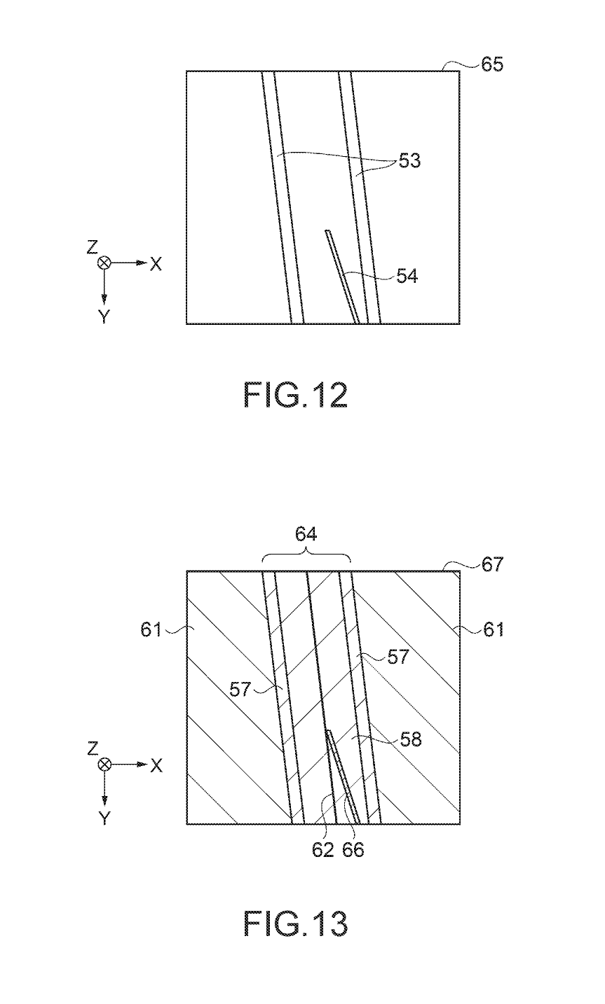

A sixth screen 65 illustrated in FIG. 12 is calculated by the image processing unit 50 on the basis of the three-dimensional ultrasonic data 43, and is a view in which the ultrasonic image is viewed from the −Z direction side. In other words, the sixth screen 65 is an image in which the blood vessel 7 and the puncture needle 8a are viewed from the emission direction 26. The sixth screen 65 is obtained by projecting the blood vessel 7 and the puncture needle 8a onto an XY plane. In addition to the blood vessel image 53, the linear needle image 54 is displayed on the sixth screen 65. The image processing unit 50 extracts the needle image 54 from the sixth screen 65. A method of extracting the needle image 54 is not particularly limited, but the needle image 54 may be calculated by calculating a difference between the sixth screen 65 and the third screen 55. The needle image 54 is an image having a grayscale from white to black, and is a gray image corresponding to the middle of black and white.

Next, as illustrated in FIG. 13, the image processing unit 50 performs a subtractive color process on the needle image 54 so as to generate a white image. The white needle image 54 is referred to as a subtractive color needle image 66 as a needle image. The image processing unit 50 combines the subtractive color needle image 66 which is extracted and subjected to the subtractive color process with the fifth screen 63 so as to calculate a seventh screen 67 as an ultrasonic image. The axis image 62 and the subtractive color needle image 66 are displayed on the seventh screen 67. The blood vessel image 64 on the seventh screen 67 is an image obtained by cutting the blood vessel 7 at a plane intersecting the emission direction 26 along the axis of the blood vessel 7. The blood vessel image 64 includes the axis image 62 indicating the axis of the blood vessel 7. The seventh screen 67 is a screen displayed on the second display device 10, and is one of screens displayed on the first display device 9. The operator can easily cause the tip of the puncture needle 8a to come close to the axis of the blood vessel 7 by viewing the seventh screen 67.

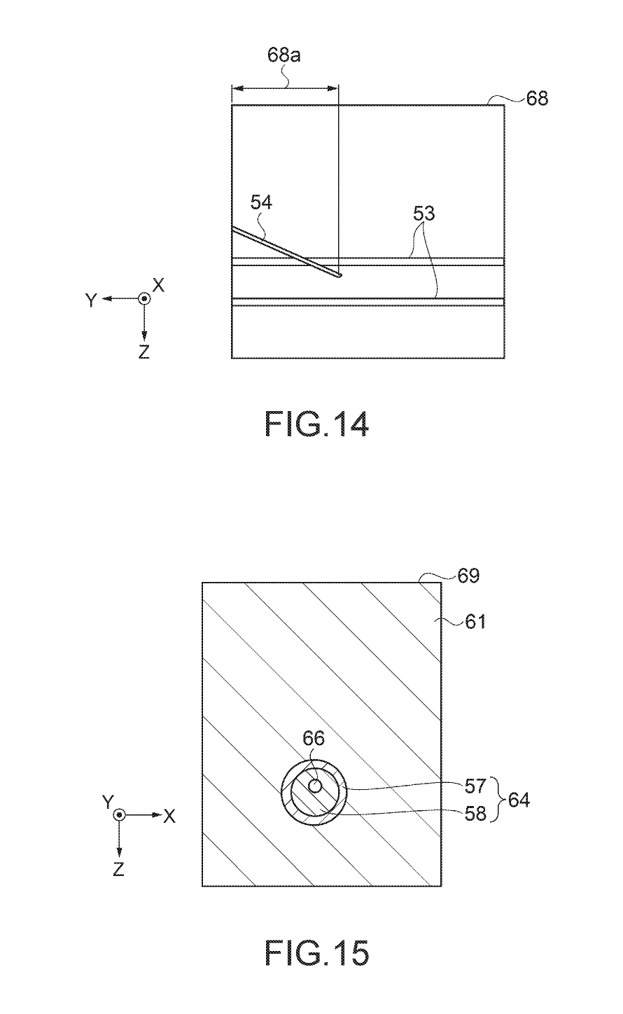

An eighth screen 68 illustrated in FIG. 14 is an image in which the blood vessel 7 and the syringe 8 are viewed from the X direction side, and is referred to as a B mode image. The eighth screen 68 includes the blood vessel image 53 which is an image of the blood vessel 7 and the needle image 54 which is an image of the puncture needle 8a. The image processing unit 50 calculates a position of a front end of the needle image 54 in the Y direction. Specifically, a length 68a from an end of the eighth screen 68 on the Y direction side to the front end of the needle image 54 is calculated.

Next, as illustrated in FIG. 15, the image processing unit 50 performs a subtractive color process such as binarization on an image of an XZ plane having the length 68a from the end on the +Y direction side, so as to calculate a ninth screen 69 as an ultrasonic image in which each region is displayed in a predetermined color. A color of each region is the same as that in the fourth screen 56 or the seventh screen 67, and the subtractive color needle image 66 is set to be white. The ninth screen 69 displays the blood vessel image 64 formed of the blood vessel wall images 57 and the blood vessel inside image 58. The blood vessel inside image 58 is displayed in an approximately circular shape, and the blood vessel wall images 57 are displayed in a ring shape. The subtractive color needle image 66 and the tissue image 61 are also displayed. The blood vessel image 64 on the ninth screen 69 is an image obtained by cutting the blood vessel 7 at a plane intersecting the axis of the blood vessel 7 and passing in the emission direction 26 of the ultrasonic wave 23. The subtractive color needle image 66 is an image in which the tip of the puncture needle 8a is viewed from the axial direction of the blood vessel 7. The ninth screen 69 is one of screens displayed on the first display device 9. The operator can check relative positions between the blood vessel 7 and the puncture needle 8a viewed from the axial direction of the blood vessel 7 by viewing the ninth screen 69. As a result, the operator can more easily recognize a position of the puncture needle 8a relative to the blood vessel 7.

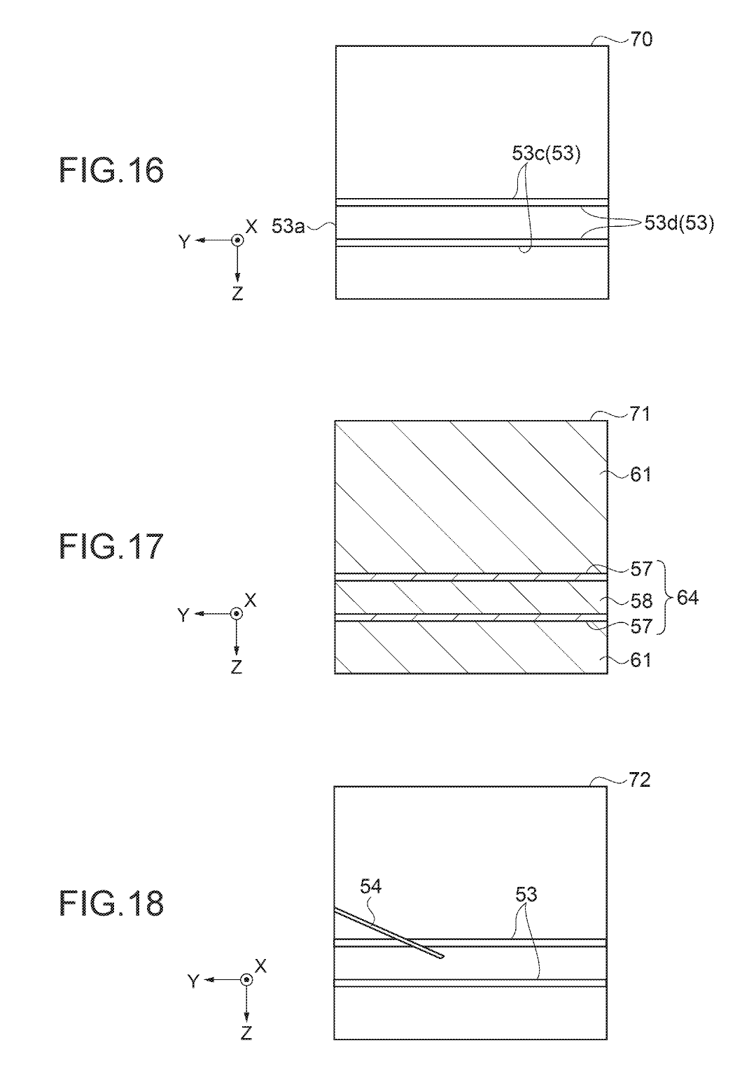

A second virtual line 53e which passes through the center 53a of the blood vessel in the first screen 52 illustrated in FIG. 8 and extends in the Z direction is set. The image processing unit 50 calculates a coordinate of the center of the blood vessel image 53 on the second screen at the end on the −Y direction side. Next, a second virtual line passing through the center 53a in the second screen and extending in the Z direction is set. The image processing unit 50 provides a second virtual plane which passes through the second virtual line 53e in the first screen 52 and the second virtual line in the second screen and is directed toward the X direction side. Next, as illustrated in FIG. 16, the image processing unit 50 calculates a tenth screen 70 in which the second virtual plane is viewed from the X direction side. The blood vessel image 53 is displayed on the tenth screen 70. The outer wall image 53c indicating the outer wall of the blood vessel 7 is displayed in the blood vessel image 53. The inner wall image 53d indicating the inner wall of the blood vessel 7 is displayed in the blood vessel image 53. The blood vessel image 53 on the tenth screen 70 is an image obtained by cutting the blood vessel 7 at a plane passing in the emission direction 26 along the axis of the blood vessel 7.

Next, as illustrated in FIG. 17, the image processing unit 50 performs a subtractive color process such as binarization on the tenth screen 70 so as to calculate an eleventh screen 71 in which each region is displayed in a predetermined color. A color set in each region is the same as the color of the fourth screen 56, the seventh screen 67, and the ninth screen 69. The blood vessel image 64 formed of the blood vessel wall images 57 and the blood vessel inside image 58 is displayed on the eleventh screen 71.

A twelfth screen 72 illustrated in FIG. 18 is calculated by the image processing unit 50 on the basis of the three-dimensional ultrasonic data 43, and is a view in which the inside of the arm 6a is viewed from the +X direction side. In other words, the twelfth screen 72 is an image in which the blood vessel 7 and the puncture needle 8a are viewed from the direction orthogonal to the emission direction 26 and the blood vessel axis direction 27. The twelfth screen 72 is obtained by projecting the blood vessel 7 and the puncture needle 8a onto an YZ plane. In addition to the blood vessel image 53, the linear needle image 54 is displayed on the twelfth screen 72. The image processing unit 50 extracts the needle image 54 from the twelfth screen 72. A method of extracting the needle image 54 is not particularly limited, but the needle image 54 may be calculated by calculating a difference between the twelfth screen 72 and the tenth screen 70.

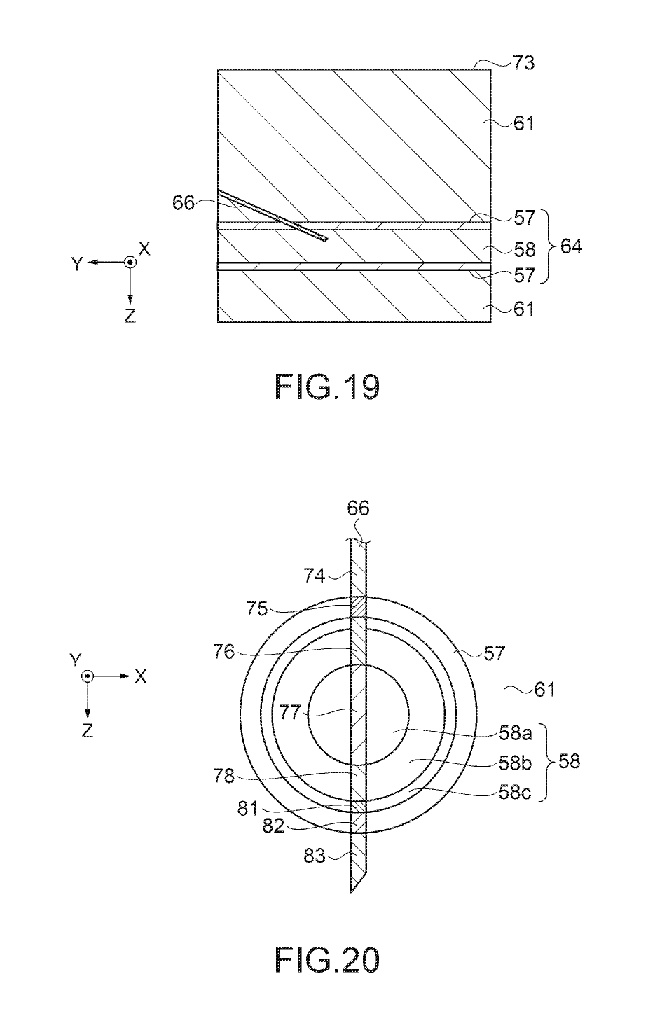

Next, as illustrated in FIG. 19, the image processing unit 50 calculates the subtractive color needle image 66 which is white by extracting the needle image 54. The image processing unit 50 combines the subtractive color needle image 66 which is white with the eleventh screen 71 so as to calculate a thirteenth screen 73 as an ultrasonic image. The blood vessel image 64 and the subtractive color needle image 66 are displayed on the thirteenth screen 73. The blood vessel image 64 is an image obtained by cutting the blood vessel 7 at a plane passing in the emission direction 26 along the axis of the blood vessel 7. The subtractive color needle image 66 is an image in which the puncture needle 8a is viewed from a direction intersecting the axial direction of the blood vessel 7 and intersecting the emission direction 26. The thirteenth screen 73 is one of screens displayed on the first display device 9. The operator can check relative positions between the blood vessel 7 and the puncture needle 8a viewed from the direction intersecting the axial direction of the blood vessel 7 and the emission direction 26 by viewing the thirteenth screen 73. As a result, the operator can more easily recognize a position of the puncture needle 8a relative to the blood vessel 7.

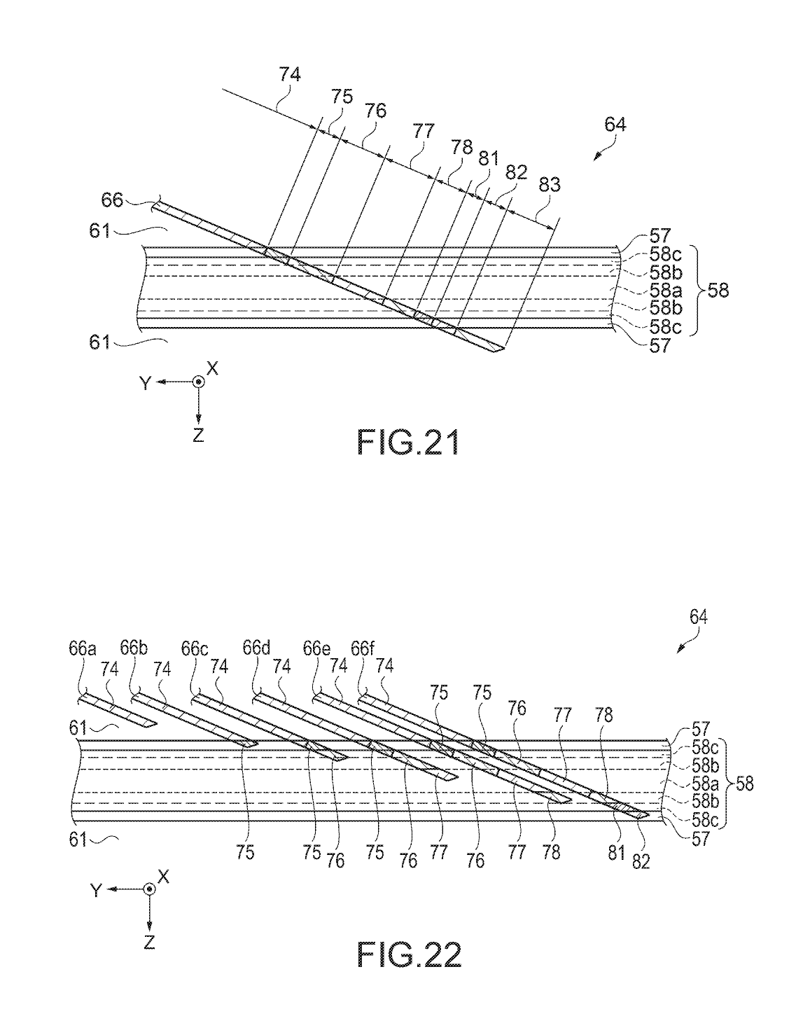

FIGS. 20 and 21 are schematic diagrams for explaining colors of the subtractive color needle image 66. FIG. 20 is a view in which the blood vessel 7 is viewed from the Y direction side, and FIG. 21 is a view in which the blood vessel 7 is viewed from the X direction side. As illustrated in FIGS. 20 and 21, the image processing unit 50 sets a color of the subtractive color needle image 66 to white, and colors and displays the subtractive color needle image 66. A pattern of a color is not particularly limited. In the present embodiment, for example, the subtractive color needle image 66 is displayed in several colors among a first color 74 to an eighth color 83. A color of the subtractive color needle image 66 is separated in a region through which the puncture needle 8a passes. The region is a region indicated by the tissue image 61, the blood vessel wall images 57, and the blood vessel inside image 58.

The region indicated by the blood vessel inside image 58 is further divided into a blood vessel inside central portion 58a, a blood vessel inside outer circumferential portion 58b, and a blood vessel inside outermost circumferential portion 58c. The blood vessel inside central portion 58a is a region in which a distance from the center of the blood vessel inside image 58 is less than 50% of the radius thereof. The blood vessel inside outer circumferential portion 58b is a region in which a distance from the center of the blood vessel inside image 58 is equal to or more than 50% and less than 90% of the radius thereof. The blood vessel inside outermost circumferential portion 58c is a region in which a distance from the center of the blood vessel inside image 58 is equal to or more than 90% and equal to or less than 100% of the radius thereof.

In a case where the puncture needle 8a does not reach the blood vessel 7, the subtractive color needle image 66 is displayed white which is the first color 74. The tissue image 61 as the background is gray, and thus the subtractive color needle image 66 can be identified. The subtractive color needle image 66 is displayed blue which is the second color 75 at the location of the blood vessel wall images 57. The blood vessel wall images 57 as the background is white, and thus the subtractive color needle image 66 can be identified. Colors of the subtractive color needle image 66 are different from each other at a location where the puncture needle 8a is not inserted into the blood vessel 7 and a location where the puncture needle 8a is inserted into the blood vessel 7. The operator can easily recognize a location where the tip of the puncture needle 8a is inserted into the blood vessel 7 by viewing the colors of the subtractive color needle image 66.

In a case where the puncture needle 8a does not reach the center of the blood vessel 7, the subtractive color needle image 66 is displayed light blue which is the third color 76 at the locations of the blood vessel inside outermost circumferential portion 58c and the blood vessel inside outer circumferential portion 58b. The subtractive color needle image 66 is displayed green which is the fourth color 77 at the location of the blood vessel inside central portion 58a. The subtractive color needle image 66 is displayed yellowish green which is the fifth color 78 at the location of the blood vessel inside outer circumferential portion 58b in a case where the subtractive color needle image 66 has passed through the blood vessel inside central portion 58a. The subtractive color needle image 66 is displayed yellow which is the sixth color 81 at the location of the blood vessel inside outermost circumferential portion 58c in a case where the subtractive color needle image 66 has passed through the blood vessel inside central portion 58a. The blood vessel inside image 58 as the background is black, and thus the subtractive color needle image 66 in the third color 76, the fourth color 77, the fifth color 78, and the sixth color 81 can be identified.

The subtractive color needle image 66 is displayed orange which is the seventh color 82 at the location of the blood vessel wall images 57 in a case where the subtractive color needle image 66 has passed through the blood vessel inside central portion 58a. The subtractive color needle image 66 is displayed red which is the eighth color 83 at the location where the subtractive color needle image 66 has broken through the blood vessel wall images 57 from the blood vessel inside central portion 58a side and has reached the tissue image 61. Colors which are conspicuous stepwise from the fifth color 78 to the eighth color 83 are set. A color of the subtractive color needle image 66 is changed from a cold color to a warm color.

The first color 74 to the eighth color 83 are preferably selected from among colors included in 8-bit colors, that is, 256 colors. When a color is generated by using red, green, and blue (RGB), 8 grayscales of 3 bits are assigned to the R and G components, and 4 grayscales of 2 bits are assigned to the B components. In this method, it is possible to reduce a data amount for storing color data.

FIG. 22 illustrates aspects of colors of the subtractive color needle image 66 from before the subtractive color needle image 66 enters the blood vessel image 64 until the subtractive color needle image 66 comes out of the blood vessel inside image 58. As illustrated in FIG. 22, a first needle image 66a does not reach the blood vessel image 64. Thus, the first needle image 66a is displayed in the first color 74. A front end of a second needle image 66b reaches the blood vessel wall images 57. Thus, the front end of the second needle image 66b is displayed in the second color 75. A front end of a third needle image 66c reaches the blood vessel inside outer circumferential portion 58b. Thus, the front end of the third needle image 66c is displayed in the third color 76. In the third needle image 66c, the first color 74, the second color 75, and the third color 76 are displayed in stripe patterns.

A front end of a fourth needle image 66d reaches the blood vessel inside central portion 58a. Thus, the front end of the fourth needle image 66d is displayed in the fourth color 77. The operator operates the syringe 8 so that the front end of the subtractive color needle image 66 is maintained in the fourth color 77. A front end of a fifth needle image 66e reaches the blood vessel inside outer circumferential portion 58b. Thus, the front end of the fifth needle image 66e is displayed in the fifth color 78. A front end of a sixth needle image 66f reaches the blood vessel wall images 57. Thus, the front end of the sixth needle image 66f is displayed in the seventh color 82. As illustrated in FIG. 21, when the front end of the subtractive color needle image 66 breaks through the blood vessel wall images 57, the front end of the subtractive color needle image 66 is displayed in the eighth color 83. The subtractive color needle image 66 is displayed in the colors corresponding to the respective regions of the blood vessel image 64.

Color display of the subtractive color needle image 66 is performed on the respective screens such as the seventh screen 67, the ninth screen 69, and the thirteenth screen 73. As mentioned above, predetermined colors corresponding to relative positions between the blood vessel 7 and the puncture needle 8a are displayed in the subtractive color needle image 66. The operator can easily recognize a position of the puncture needle 8a relative to the blood vessel 7 by viewing the subtractive color needle image 66. Since the operator has only to look at the subtractive color needle image 66, the operator can more easily recognize a position of the puncture needle 8a relative to the blood vessel 7 than in a case where the operator looks at a plurality of locations.

Colors of the subtractive color needle image 66 are different from each other at a location where the puncture needle 8a is not inserted into the blood vessel 7 and a location where the puncture needle 8a is inserted into the blood vessel 7. The operator can easily recognize a location where the tip of the puncture needle 8a is inserted into the blood vessel 7 by viewing the colors of the subtractive color needle image 66.

Colors of the subtractive color needle image 66 are different from each other at a location where the puncture needle 8a has not passed through the center of the blood vessel 7 and a location where the puncture needle 8a has passed through the center of the blood vessel 7. At the portion where the puncture needle 8a has not passed through the center of the blood vessel 7, a color of the subtractive color needle image 66 is changed to the first color 74, the second color 75, and the third color 76. At the portion where the puncture needle 8a has passed through the center of the blood vessel 7, a color of the subtractive color needle image 66 is changed to the fifth color 78, the sixth color 81, the seventh color 82, and the eighth color 83. The operator can easily judge whether or not the tip of the puncture needle 8a passes through the center of the blood vessel 7 by viewing the subtractive color needle image 66.

Colors of the subtractive color needle image 66 in the blood vessel inside image 58 are the third color 76 to the sixth color 81, and a color of the blood vessel inside image 58 is black. Therefore, since a color of the subtractive color needle image 66 has brightness or saturation higher than that of a color of the blood vessel inside image 58, and thus it is possible to easily judge whether or not the subtractive color needle image 66 is located inside the blood vessel inside image 58.

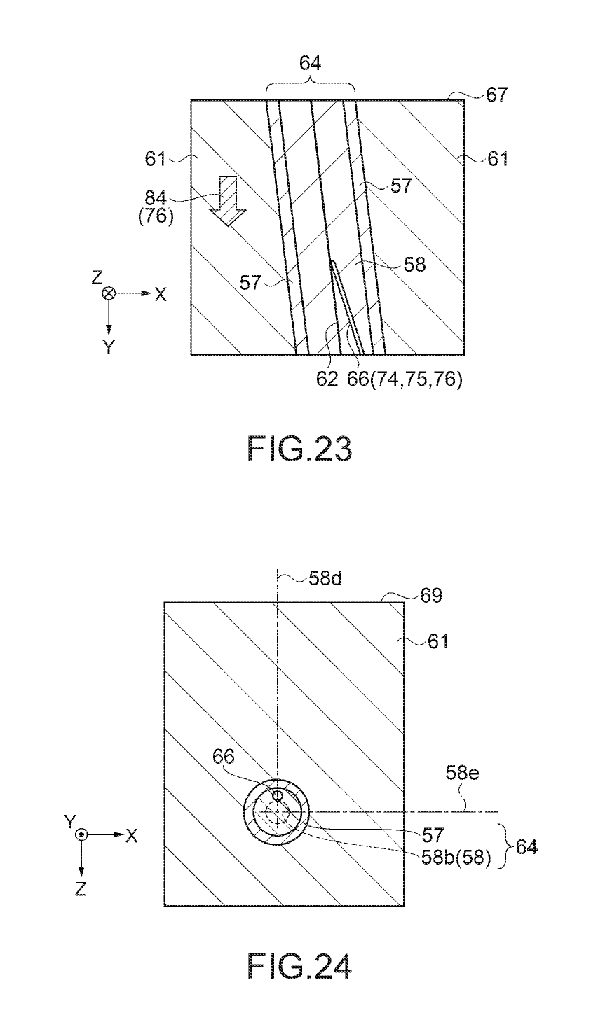

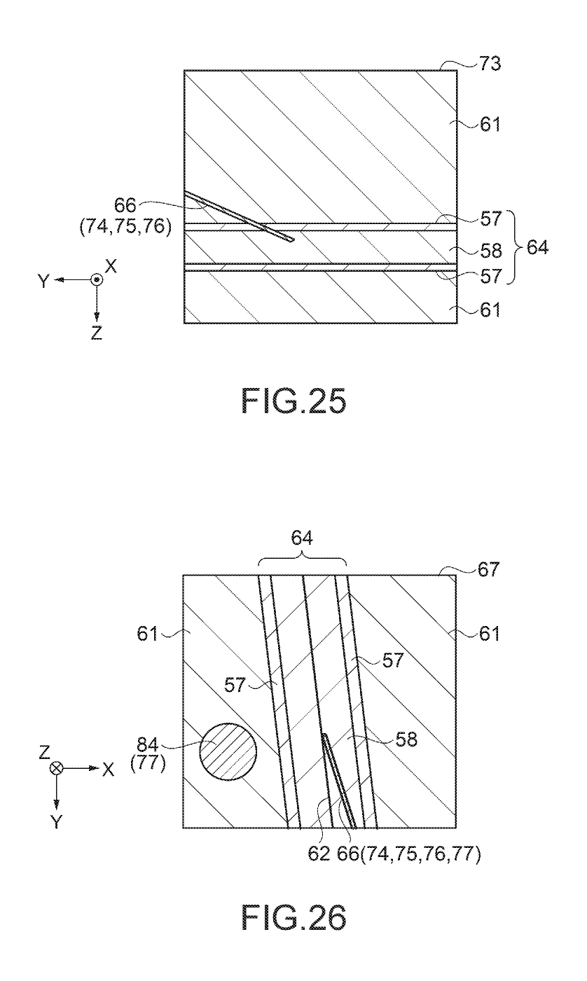

FIGS. 23 to 37 are schematic diagrams for explaining a guide mark. FIGS. 23 to 25 are diagrams in a case where the front end of the subtractive color needle image 66 does not reach the blood vessel inside central portion 58a, and is located at the blood vessel inside outer circumferential portion 58b, in which FIG. 23 illustrates the seventh screen 67, FIG. 24 illustrates the ninth screen 69, and FIG. 25 illustrates the thirteenth screen 73. The first display device 9 displays the seventh screen 67, the ninth screen 69, and the thirteenth screen 73, and the second display device 10 displays the seventh screen 67. Relative positions between the blood vessel image 64 and the subtractive color needle image 66 can be easily understood by referring to the seventh screen 67, the ninth screen 69, and the thirteenth screen 73 on the first display device 9, and thus the operator can easily understand relative positions between the blood vessel 7 and the puncture needle 8a.

As illustrated in FIG. 23, the front end of the subtractive color needle image 66 is located near the axis image 62 in the seventh screen 67. A guide mark 84 as a mark image is displayed in the seventh screen 67. The guide mark 84 guides a direction in which the tip of the puncture needle 8a comes close to the axis of the blood vessel 7. The operator can easily cause the tip of the puncture needle 8a to come close to the axis of the blood vessel 7 by viewing the guide mark 84.

The guide mark 84 in the figure is an arrow indicating the lower side. The arrow indicating a lower side indicates a guide to move the tip of the puncture needle 8a to a deeper side. In other words, the arrow indicates the +Z direction. If the guide mark 84 is an arrow indicating the right side in the figure, the guide mark 84 indicates a guide to move the tip of the puncture needle 8a in the +X direction. If the guide mark 84 is an arrow indicating the left side in the figure, the guide mark 84 indicates a guide to move the tip of the puncture needle 8a in the −X direction. If the guide mark 84 does not indicate the right and left sides, the guide mark 84 indicates that the tip of the puncture needle 8a is not moved in the X direction.

A position of the front end of the subtractive color needle image 66 is located near the axis image 62. As illustrated in FIG. 24, the subtractive color needle image 66 is located on a first central line 58d which is a central line of the blood vessel inside image 58 in the X direction. At this time, the guide direction calculation unit 51 determines that a guide not to move the puncture needle 8a in the X direction is displayed. As illustrated in FIG. 23, an arrow indicating the left or the right is not displayed in the guide mark 84.

A color of the guide mark 84 is the third color 76. A color of the guide mark 84 is the same as a color of the front end of the subtractive color needle image 66. In the seventh screen 67 and the thirteenth screen 73, colors of the subtractive color needle image 66 are disposed in stripe patterns in order of the first color 74, the second color 75, and the third color 76. In the seventh screen 67, the ninth screen 69, and the thirteenth screen 73, a color of the front end of the subtractive color needle image 66 is the third color 76.

As illustrated in FIG. 24, the subtractive color needle image 66 is located further toward the −Z direction side than a second central line 58e which is a central line of the blood vessel inside image 58 in the Z direction. At this time, the guide direction calculation unit 51 determines that a guide to move the puncture needle 8a in the +Z direction is displayed. As illustrated in FIG. 23, the arrow indicating the lower side in the figure is displayed in the guide mark 84. The operator checks the guide mark 84 in the seventh screen 67 displayed on the second display device 10. Since the guide mark 84 and the syringe 8 are disposed to be close to each other, the operator can check a direction in which the puncture needle 8a is to be moved through short visual line movement.

As illustrated in FIG. 25, it is possible to easily understand relative positions in the Z direction between the blood vessel image 64 and the subtractive color needle image 66 from the thirteenth screen 73. Therefore, the operator can easily understand to what extent the tip of the puncture needle 8a is preferably moved in the +Z direction side.

FIGS. 26 to 28 respectively illustrate the seventh screen 67, the ninth screen 69, and the thirteenth screen 73 in a case where the front end of the subtractive color needle image 66 is located at the blood vessel inside central portion 58a. As illustrated in FIG. 26, the front end of the subtractive color needle image 66 is located near the axis image 62 in the seventh screen 67. The guide mark 84 is displayed in the seventh screen 67. The guide mark 84 is a round mark, and indicates that the tip of the puncture needle 8a is not required to be moved.

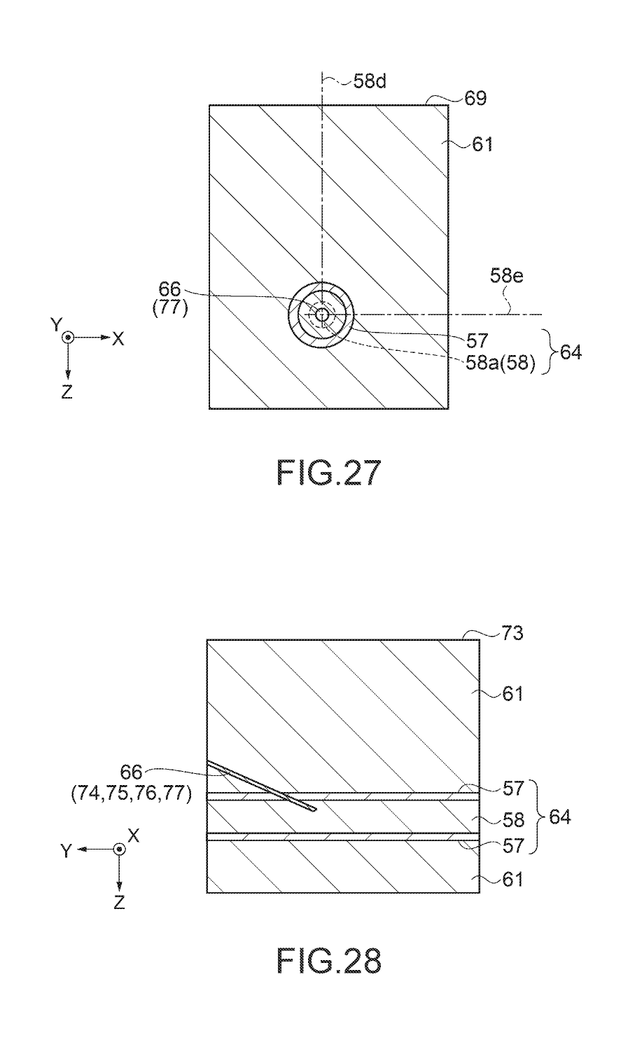

The front end of the subtractive color needle image 66 is located near the axis image 62. As illustrated in FIG. 27, the subtractive color needle image 66 is located on the first central line 58d which is a central line of the blood vessel inside image 58 in the X direction. At this time, the guide direction calculation unit 51 determines that a guide not to move the puncture needle 8a in the X direction is displayed. An arrow indicating the left or the right is not displayed in the guide mark 84. A color of the guide mark 84 is the fourth color 77. In the seventh screen 67 and the thirteenth screen 73, colors of the subtractive color needle image 66 are disposed in stripe patterns in order of the first color 74, the second color 75, the third color 76, and the fourth color 77. In the seventh screen 67, the ninth screen 69, and the thirteenth screen 73, a color of the front end of the subtractive color needle image 66 is the fourth color 77.

As illustrated in FIG. 27, the subtractive color needle image 66 comes close to the second central line 58e which is a central line of the blood vessel inside image 58 in the Z direction. At this time, the guide direction calculation unit 51 determines that a guide not to move the puncture needle 8a in the Z direction is displayed. As illustrated in FIG. 26, a round mark is displayed in the guide mark 84. The operator checks the guide mark 84 in the seventh screen 67 displayed on the second display device 10.

As illustrated in FIG. 28, it is possible to easily understand relative positions in the Z direction between the blood vessel image 64 and the subtractive color needle image 66 from the thirteenth screen 73. The operator can easily understand that the front end of the subtractive color needle image 66 is located at the center of the blood vessel inside image 58.

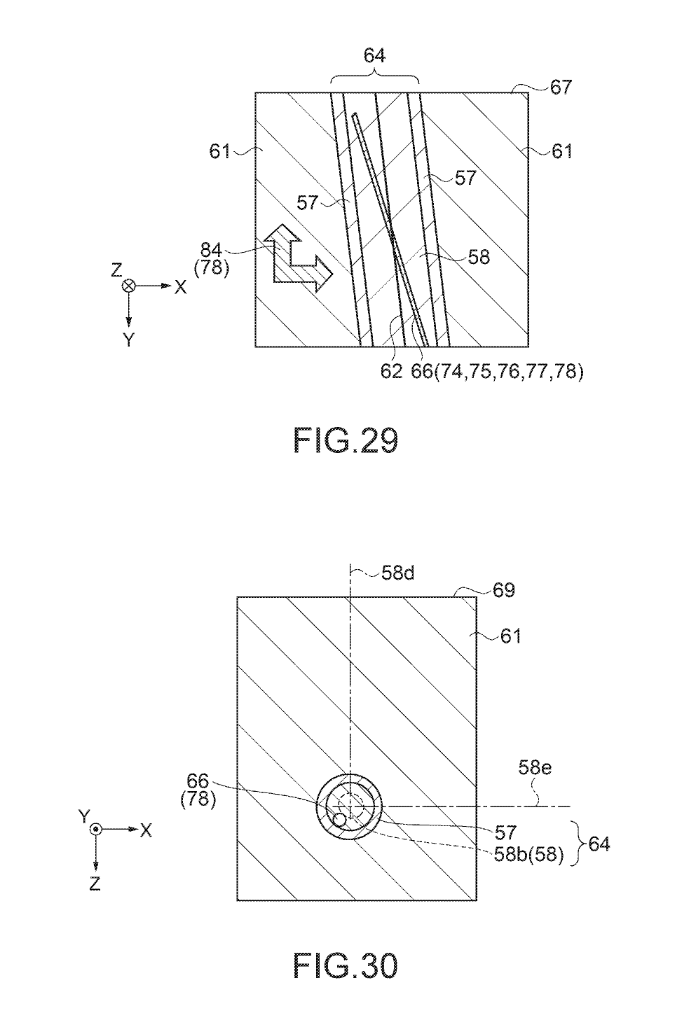

FIGS. 29 to 31 respectively illustrate the seventh screen 67, the ninth screen 69, and the thirteenth screen 73 in a case where the front end of the subtractive color needle image 66 has passed through the blood vessel inside central portion 58a and is located at the blood vessel inside outer circumferential portion 58b. As illustrated in FIG. 29, the front end of the subtractive color needle image 66 is located on the −X direction side of the axis image 62 in the seventh screen 67. The guide mark 84 has an arrow indicating the right side in the figure, and indicates a guide to move the tip of the puncture needle 8a to the +X direction side.

The front end of the subtractive color needle image 66 is located on the −X direction side of the axis image 62. As illustrated in FIG. 30, the subtractive color needle image 66 is located further toward the −X direction side than the first central line 58d which is a central line of the blood vessel inside image 58 in the X direction. At this time, the guide direction calculation unit 51 determines that a guide to move the puncture needle 8a in the +X direction is displayed. As illustrated in FIG. 29, the arrow indicating the right side in the figure is displayed in the guide mark 84. A color of the guide mark 84 is the fifth color 78. In the seventh screen 67 and the thirteenth screen 73, colors of the subtractive color needle image 66 are disposed in stripe patterns in order of the first color 74, the second color 75, the third color 76, the fourth color 77, and the fifth color 78. In the seventh screen 67, the ninth screen 69, and the thirteenth screen 73, a color of the front end of the subtractive color needle image 66 is the fifth color 78.

As illustrated in FIG. 30, the subtractive color needle image 66 is located further toward the +Z direction side than the second central line 58e which is a central line of the blood vessel inside image 58 in the Z direction. At this time, the guide direction calculation unit 51 determines that a guide to move the puncture needle 8a to the −Z direction side is displayed. As illustrated in FIG. 29, an arrow indicating the upper side in the figure is displayed in the guide mark 84. The operator checks the guide mark 84 in the seventh screen 67 displayed on the second display device 10.

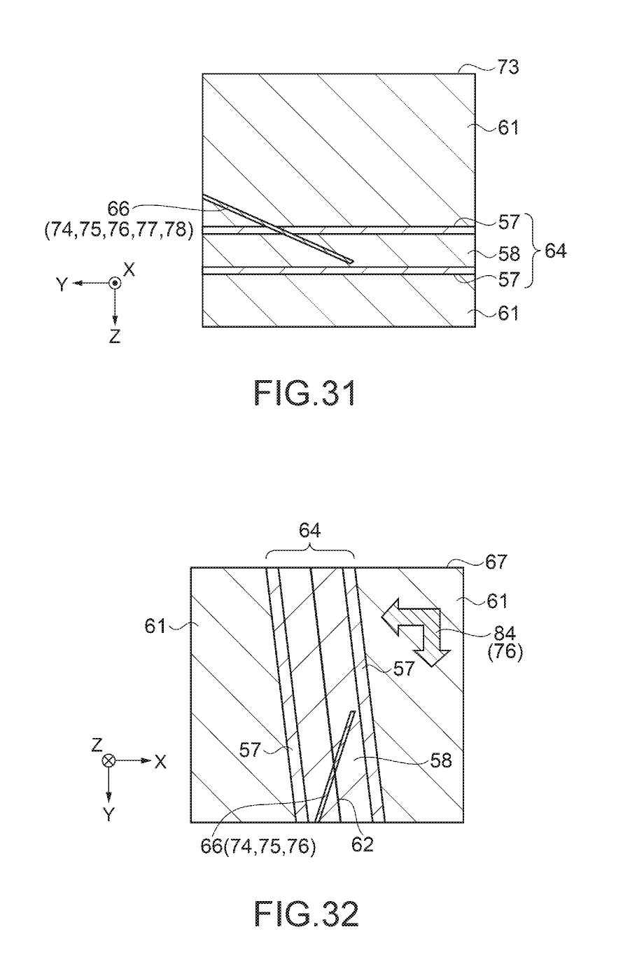

As illustrated in FIG. 31, it is possible to easily understand relative positions in the Z direction between the blood vessel image 64 and the subtractive color needle image 66 from the thirteenth screen 73. The operator can easily understand to what extent the tip of the puncture needle 8a is preferably moved to the −Z direction side.

FIGS. 32 to 34 respectively illustrate the seventh screen 67, the ninth screen 69, and the thirteenth screen 73 in a case where the front end of the subtractive color needle image 66 does not reach the blood vessel inside central portion 58a and is located at the blood vessel inside outer circumferential portion 58b. As illustrated in FIG. 32, the front end of the subtractive color needle image 66 is located on the +X direction side of the axis image 62 in the seventh screen 67. The guide mark 84 is an arrow indicating the left side in the figure, and indicates a guide to move the tip of the puncture needle 8a to the −X direction side.

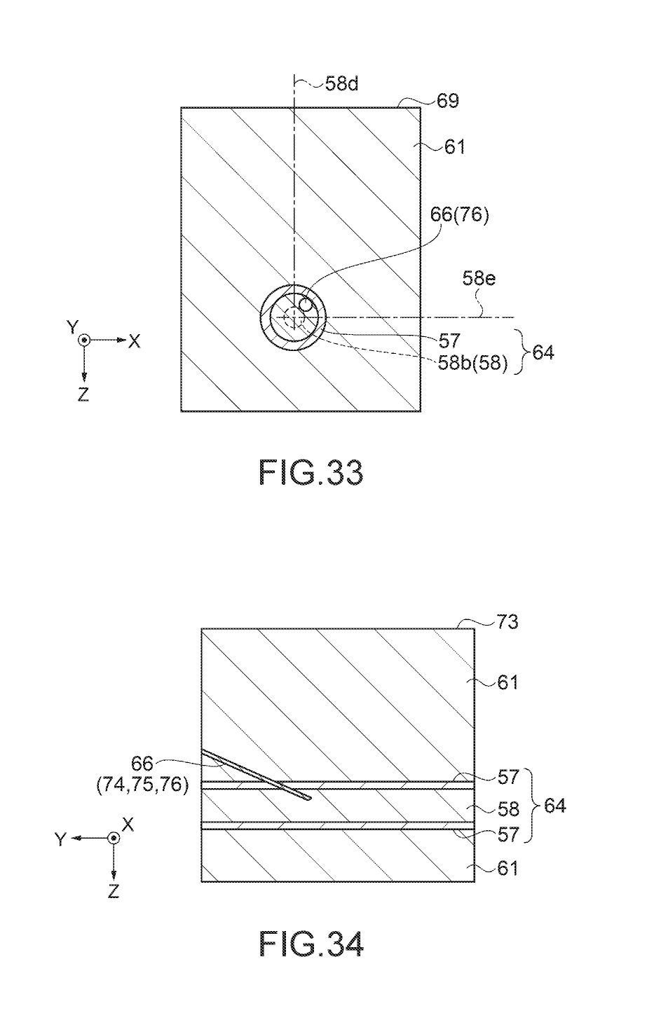

The front end of the subtractive color needle image 66 is located on the +X direction side of the axis image 62. As illustrated in FIG. 33, the subtractive color needle image 66 is located further toward the +X direction side than the first central line 58d which is a central line of the blood vessel inside image 58 in the X direction. At this time, the guide direction calculation unit 51 determines that a guide to move the puncture needle 8a in the −X direction is displayed. As illustrated in FIG. 32, the arrow indicating the left side in the figure is displayed in the guide mark 84. A color of the guide mark 84 is the third color 76. In the seventh screen 67 and the thirteenth screen 73, colors of the subtractive color needle image 66 are disposed in stripe patterns in order of the first color 74, the second color 75, and the third color 76. In the seventh screen 67, the ninth screen 69, and the thirteenth screen 73, a color of the front end of the subtractive color needle image 66 is the third color 76.

As illustrated in FIG. 33, the subtractive color needle image 66 is located further toward the −Z direction side than the second central line 58e which is a central line of the blood vessel inside image 58 in the Z direction. At this time, the guide direction calculation unit 51 determines that a guide to move the puncture needle 8a to the +Z direction side is displayed. As illustrated in FIG. 32, an arrow indicating the lower side in the figure is displayed in the guide mark 84. The operator checks the guide mark 84 in the seventh screen 67 displayed on the second display device 10.

As illustrated in FIG. 34, it is possible to easily understand relative positions in the Z direction between the blood vessel image 64 and the subtractive color needle image 66 from the thirteenth screen 73. The operator can easily understand to what extent the tip of the puncture needle 8a is preferably moved to the +Z direction side.

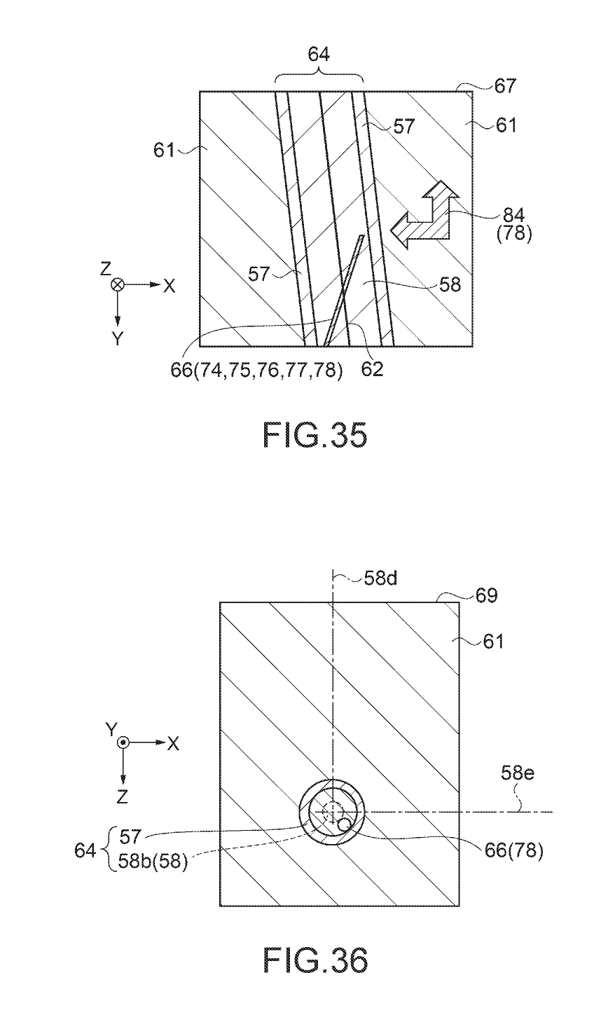

FIGS. 35 to 37 respectively illustrate the seventh screen 67, the ninth screen 69, and the thirteenth screen 73 in a case where the front end of the subtractive color needle image 66 has passed through the blood vessel inside central portion 58a and is located at the blood vessel inside outer circumferential portion 58b. As illustrated in FIG. 35, the front end of the subtractive color needle image 66 is located on the +X direction side of the axis image 62 in the seventh screen 67. The guide mark 84 is an arrow indicating the left side in the figure, and indicates a guide to move the tip of the puncture needle 8a to the −X direction side.

The front end of the subtractive color needle image 66 is located on the +X direction side of the axis image 62. As illustrated in FIG. 36, the subtractive color needle image 66 is located further toward the +X direction side than the first central line 58d which is a central line of the blood vessel inside image 58 in the X direction. At this time, the guide direction calculation unit 51 determines that a guide to move the puncture needle 8a in the −X direction is displayed. As illustrated in FIG. 35, the arrow indicating the left side in the figure is displayed in the guide mark 84. A color of the guide mark 84 is the fifth color 78. In the seventh screen 67 and the thirteenth screen 73, colors of the subtractive color needle image 66 are disposed in stripe patterns in order of the first color 74, the second color 75, the third color 76, the fourth color 77, and the fifth color 78. In the seventh screen 67, the ninth screen 69, and the thirteenth screen 73, a color of the front end of the subtractive color needle image 66 is the fifth color 78.

The seventh screen 67 include the guide mark 84 for indicating a direction in which the front end of the subtractive color needle image 66 comes close to the axis image 62. The operator can easily judge in which direction the tip of the puncture needle 8a is preferably moved so the tip of the puncture needle 8a comes close to the axis of the blood vessel 7 by viewing the guide mark 84. Therefore, the operator can easily cause the tip of the puncture needle 8a to come close to the axis of the blood vessel 7.

As illustrated in FIG. 36, the subtractive color needle image 66 is located further toward the +Z direction side than the second central line 58e which is a central line of the blood vessel inside image 58 in the Z direction. At this time, the guide direction calculation unit 51 determines that a guide to move the puncture needle 8a to the −Z direction side is displayed. As illustrated in FIG. 35, an arrow indicating the upper side in the figure is displayed in the guide mark 84. The operator checks the guide mark 84 in the seventh screen 67 displayed on the second display device 10.

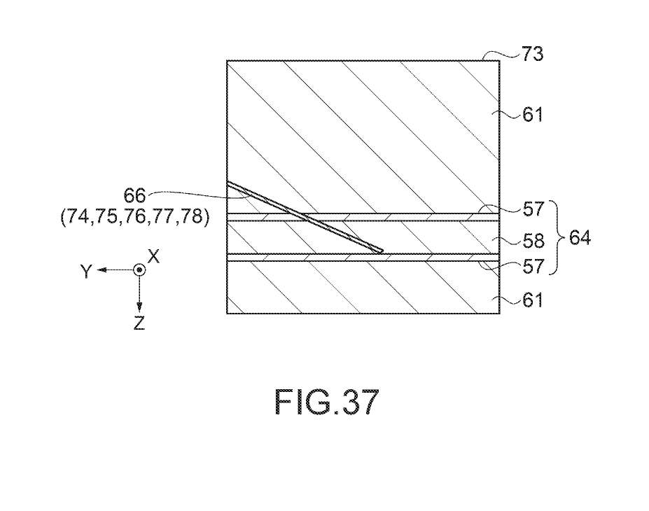

As illustrated in FIG. 37, it is possible to easily understand relative positions in the Z direction between the blood vessel image 64 and the subtractive color needle image 66 from the thirteenth screen 73. The operator can easily understand to what extent the tip of the puncture needle 8a is preferably moved to the −Z direction side.

In the display process instep S4, the first display device 9 provided in the control device 3 displays the seventh screen 67, the ninth screen 69, and the thirteenth screen 73. The second display device 10 provided in the ultrasonic probe 2 displays the seventh screen 67. The second display device 10 is provided on the arm 6a, and is located near the puncture needle 8a. An image in which the blood vessel 7 and the puncture needle 8a are viewed from the second display device 10 side is displayed on the seventh screen 67. The axis image 62 and the guide mark 84 are displayed on the seventh screen 67. The operator can easily recognize a direction in which the tip of the puncture needle 8a is to be moved by viewing the second display device 10.

The seventh screen 67, the ninth screen 69, and the thirteenth screen 73 displayed on the first display device 9 are views in which the blood vessel 7 and the puncture needle 8a are viewed from three different directions. The operator can easily understand relative positions between the blood vessel 7 and the puncture needle 8a by viewing the first display device 9. Therefore, the operator can reliably insert the puncture needle 8a into the center of the blood vessel 7.

In the finish determination process in step S5, the operator checks whether or not the puncture needle 8a can be inserted into the center of the blood vessel 7 while viewing the first display device 9 and the second display device 10. In a case where the puncture needle 8a is not inserted into the center of the blood vessel 7, step S1 to step S4 are repeatedly performed. In a case where the puncture needle 8a can be inserted into the center of the blood vessel 7, the process of inserting the puncture needle 8a into the blood vessel 7 is finished.

As described above, according to the present embodiment, the following effects are achieved.

(1) According to the present embodiment, the ultrasonic measurement apparatus 1 includes the ultrasonic probe 2, the image processing unit 50, the first display device 9, and the second display device 10. The ultrasonic probe 2 emits the ultrasonic waves 23 to the arm 6a, detects reflected waves from the arm 6a, and outputs ultrasonic signals. The image processing unit 50 forms the seventh screen 67, the ninth screen 69, and the thirteenth screen 73 on the basis of the ultrasonic signals, the first display device 9 displays the seventh screen 67, the ninth screen 69, and the thirteenth screen 73, and the second display device 10 displays the seventh screen 67. Each of the seventh screen 67, the ninth screen 69, and the thirteenth screen 73 includes the blood vessel image 64 and the subtractive color needle image 66. The blood vessel 7 into which the puncture needle 8a is inserted is present in the arm 6a. An operator inserts the puncture needle 8a into the blood vessel 7. The blood vessel image 64 is an image indicating the blood vessel 7. The subtractive color needle image 66 is an image indicating the puncture needle 8a inserted into the arm 6a. Predetermined colors corresponding to relative positions between the blood vessel 7 and the puncture needle 8a are displayed in the seventh screen 67, the ninth screen 69, and the thirteenth screen 73. As a result, the operator can easily recognize a position of the puncture needle 8a relative to the blood vessel 7 by viewing the seventh screen 67, the ninth screen 69, and the thirteenth screen 73.

(2) According to the present embodiment, predetermined colors corresponding to relative positions between the blood vessel 7 and the puncture needle 8a are displayed in the subtractive color needle image 66. Therefore, an operator can easily recognize a position of the puncture needle 8a relative to the blood vessel 7 on the basis of a position of the subtractive color needle image 66 and color information by viewing the subtractive color needle image 66. Since the operator has only to look at the subtractive color needle image 66, the operator can more easily recognize a position of the puncture needle 8a relative to the blood vessel 7 than in a case where the operator looks at a plurality of locations.

(3) According to the present embodiment, colors of the subtractive color needle image 66 are different from each other at a location where the puncture needle 8a has not passed through the center of the blood vessel 7 and a location where the puncture needle 8a has passed through the center of the blood vessel 7. Therefore, an operator can easily judge whether or not the tip of the puncture needle 8a passes through the center of the blood vessel 7 by viewing the colors of the subtractive color needle image 66.

(4) According to the present embodiment, a color of the subtractive color needle image 66 is the first color 74 at a location where the puncture needle 8a is not inserted into the blood vessel 7, and colors of the subtractive color needle image 66 are the second color 75 to the seventh color 82 at a location where the puncture needle 8a is inserted into the blood vessel 7. Colors of the subtractive color needle image 66 are different from each other at a location where the puncture needle 8a is not inserted into the blood vessel 7 and a location where the puncture needle 8a is inserted into the blood vessel 7. Therefore, an operator can easily recognize a location where the tip of the puncture needle 8a is inserted into the blood vessel 7 by viewing the colors of the subtractive color needle image 66.

(5) According to the present embodiment, the blood vessel image 64 on the seventh screen 67 is an image obtained by cutting the blood vessel 7 at a plane intersecting the emission direction 26 of the ultrasonic wave 23 along the axis of the blood vessel 7. The blood vessel image 64 on the seventh screen 67 includes the axis image 62 indicating the axis of the blood vessel 7. When an operator inserts the puncture needle 8a into the blood vessel 7, the operator inserts the puncture needle 8a along the axis of the blood vessel 7. Since the blood vessel image 64 includes the axis image 62, the operator can insert the puncture needle 8a into the blood vessel 7 by using the axis image 62 as a guide. Therefore, the operator can easily insert the puncture needle 8a along the axis of the blood vessel 7.

(6) According to the present embodiment, the subtractive color needle image 66 on the seventh screen 67 is an image in which the puncture needle 8a is viewed from the emission direction 26 of the ultrasonic wave 23. In a case where the puncture needle 8a is taken as a predetermined section, only a part of the puncture needle 8a is displayed as an image, and thus a position of the puncture needle 8a is hardly recognized. On the other hand, the subtractive color needle image 66 on the seventh screen 67 is an image in which the puncture needle 8a is viewed from the emission direction 26, and thus the subtractive color needle image 66 shows the entire shape of the puncture needle 8a. Therefore, an operator can easily recognize relative positions between the blood vessel 7 and the puncture needle 8a.

(7) According to the present embodiment, a color of the puncture needle 8a has brightness or saturation higher than that of a color of the blood vessel inside image 58 in the seventh screen 67, the ninth screen 69, and the thirteenth screen 73. Therefore, since an operator can check the subtractive color needle image 66 in a bright color on the background in a dark color, the operator can check the subtractive color needle image 66 on an easily viewable screen.

(8) According to the present embodiment, the seventh screen 67 includes the guide mark 84 indicating a direction in which a front end of an image indicating the puncture needle 8a comes close to an image indicating the axis of the blood vessel 7. Therefore, an operator can easily cause the tip of the puncture needle 8a to come close to the axis of the blood vessel 7 by viewing the guide mark 84.

(9) According to the present embodiment, the second display device 10 is provided in the ultrasonic probe 2. Since an operator inserts the puncture needle 8a from a location close to the ultrasonic probe 2, the hands of the operator are located at the location close to the ultrasonic probe 2. Therefore, since the second display device 10 and the hands of the operator come close to each other, the operator can check the second display device 10 and the hands through short visual line movement. As a result, the operator can operate the puncture needle 8a while viewing the second display device 10, and can thus easily perform an operation of causing the tip of the puncture needle 8a to come close to an axis of the blood vessel 7.

(10) According to the present embodiment, the ninth screen 69 includes an image obtained by cutting the blood vessel 7 at a plane intersecting the axis of the blood vessel 7 and passing in the emission direction 26 of the ultrasonic wave 23. The subtractive color needle image 66 includes an image in which the puncture needle 8a is viewed from the axial direction of the blood vessel 7. The ninth screen 69 includes a view in which the blood vessel 7 and the puncture needle 8a are viewed from the axial direction of the blood vessel 7. Therefore, an operator can check relative positions between the blood vessel 7 and the puncture needle 8a viewed from the axial direction of the blood vessel 7. As a result, the operator can more easily recognize a position of the puncture needle 8a relative to the blood vessel 7.

(11) According to the present embodiment, the blood vessel image 64 on the thirteenth screen 73 is an image obtained by cutting the blood vessel 7 at a plane passing in the emission direction 26 of the ultrasonic wave 23 along the axis of the blood vessel 7. The subtractive color needle image 66 on the thirteenth screen 73 is an image in which the puncture needle 8a is viewed from a direction intersecting the axial direction of the blood vessel 7 and intersecting the emission direction 26 of the ultrasonic wave 23. The thirteenth screen 73 is a view in which the blood vessel 7 and the puncture needle 8a are viewed from a direction intersecting the axial direction of the blood vessel 7 and the emission direction 26 of the ultrasonic wave 23. Therefore, an operator can check relative positions between the blood vessel 7 and the puncture needle 8a viewed from the direction intersecting the axial direction of the blood vessel 7 and the emission direction 26 of the ultrasonic wave 23. As a result, the operator can more easily recognize a position of the puncture needle 8a relative to the blood vessel 7.

The present embodiment is not limited to the above-described embodiment, and may be variously modified or altered by a person skilled in the art. Modification examples will be described below.

### Modification Example 1

In the present embodiment, the subtractive color needle image 66 and the guide mark 84 are displayed in the first color 74 to the eighth color 83. All of the first color 74 to the eighth color 83 may not be used, and the number of colors may be reduced. For example, a color may not be changed when the subtractive color needle image 66 enters the blood vessel image 64, and a color may be changed when the subtractive color needle image 66 comes out of the blood vessel image 64. For example, colors may be displayed in locations where the subtractive color needle image 66 is located at the blood vessel inside central portion 58a and is located at the blood vessel wall images 57. Displayed colors may be changed to various combinations. The first color 74 to the eighth color 83 are respectively white, blue, light blue, green, yellowish green, orange, and red, but a specific color combination may be changed.

### Modification Example 2

In the above-described embodiment, a description has been made of an example in which the puncture needle 8a is inserted into the blood vessel 7. A bar-shaped target object into which the puncture needle 8a is inserted may be a nerve or a lymph node in addition to the blood vessel 7. Also in this case, the puncture needle 8a can be appropriately inserted into a nerve or a lymph node. The ultrasonic measurement apparatus 1 may be used in a case where an electrode needle is inserted into a tumor in a radiofrequency ablation. A target object in this case is a tumor, and an electrode needle as the puncture needle 8a is inserted into a target object. Also in this case, the puncture needle 8a can be appropriately inserted into a target object. The ultrasonic measurement apparatus 1 may be used in a case where the puncture needle 8a is inserted into an organ in a biotissue diagnosis. Also in this case, the puncture needle 8a can be reliably inserted into an organ.

### Modification Example 3

In the above-described embodiment, colors indicating relative positions between the blood vessel 7 and the puncture needle 8a are displayed in the guide mark 84 and the subtractive color needle image 66. Frames may be provided on the seventh screen 67, the ninth screen 69, and the thirteenth screen 73, and relative positions between the blood vessel 7 and the puncture needle 8a may be indicated by colors of the frames. A rectangular mark may be displayed in a screen, and relative positions between the blood vessel 7 and the puncture needle 8a may be indicated by colors of the mark. The first color 74 to the eighth color 83 may be displayed in stripe patterns in the frames or the mark.

### Modification Example 4

In the above-described embodiment, colors are displayed in the whole of the subtractive color needle image 66. Colors may be displayed in the whole or a part of the subtractive color needle image 66. A screen may be displayed in an easily viewable form.

### Modification Example 5

In the above-described embodiment, the circuit board 31 is provided with the transducer driving circuit 33. The transducer driving circuit 33 may be provided in the ultrasonic element array 17, and the circuit board 31 may be provided with an element intermediately connecting the wiring 4. A circuit configuration may be a configuration which is easily mounted.

### Modification Example 6

In the above-described embodiment, the linear motor 30 moves the ultrasonic element array 17 in the blood vessel axis direction 27. The linear motor 30 and the acoustic lens 24 may be omitted by increasing the number of transducers of the ultrasonic element array 17. Even in this case, a three-dimensional ultrasonic image can be detected.

### Modification Example 7

In the above-described embodiment, the subtractive color needle image 66 on the ninth screen 69 is an image of only the tip of the puncture needle 8a, but may be an image of the entire puncture needle 8a. An inclination of the puncture needle 8a can be checked.

### Modification Example 8

In the above-described embodiment, colors of the subtractive color needle image 66 are different from each other at a location where the puncture needle 8a has not passed through the center of the blood vessel 7 and a location where the puncture needle 8a has passed through the center of the blood vessel 7. Locations where colors of the subtractive color needle image 66 are different from each other may be set by using references other than the center of the blood vessel 7. A specific target portion in a target object such as the blood vessel 7 may be selected, and a color of the subtractive color needle image 66 may differ by using the selected target portion as a reference. In this case, it is possible to check a position of the puncture needle 8a relative to the specific target portion.

The entire disclosure of Japanese Patent Application NO. 2016-104917 filed on May 26, 2016 is expressly incorporated by reference herein.

What is claimed is:

1. An ultrasonic measurement apparatus comprising:
an ultrasonic probe that emits an ultrasonic wave to a subject, detects a reflected wave from the subject, and outputs an ultrasonic signal;
an image processing unit that forms an ultrasonic image on the basis of the ultrasonic signal; and
a display unit that displays the ultrasonic image,
wherein the ultrasonic image includes a target object image indicating a target object included in the subject, and a needle image indicating a puncture needle inserted into the target object, and
wherein predetermined colors corresponding to relative positions between the target object and the puncture needle are displayed in the ultrasonic image.

2. The ultrasonic measurement apparatus according to claim 1,
wherein a location where the predetermined colors corresponding to relative positions between the target object and the puncture needle are displayed in the ultrasonic image is the needle image.

3. the ultrasonic measurement apparatus according to claim 2,
wherein the target object has a bar shape, and
wherein colors of the needle image are different from each other at a location where the puncture needle has not passed through the center of the target object and a location where the puncture needle has passed through the center of the target object.

4. The ultrasonic measurement apparatus according to claim 2,
wherein colors of the needle image are different from each other at a location where the puncture needle is not inserted into the target object and a location where the puncture needle is inserted into the target object.

5. The ultrasonic measurement apparatus according to claim 3,
wherein the target object image is an image obtained by cutting the target object at a plane intersecting an emission direction of the ultrasonic wave along an axis of the target object, and
wherein the target object image includes an axis image indicating the axis of the target object.

6. The ultrasonic measurement apparatus according to claim 4,
wherein the needle image is an image in which the puncture needle is viewed from an emission direction of the ultrasonic wave.

7. The ultrasonic measurement apparatus according to claim 1,
wherein a color of the puncture needle has brightness or saturation higher than brightness or saturation of a color of the inside of the target object.

8. The ultrasonic measurement apparatus according to claim 3,
wherein the ultrasonic image includes a mark image indicating a direction in which a front end of the needle image comes close to an axis of the target object image.

9. The ultrasonic measurement apparatus according to claim 1,
wherein the display unit is provided in the ultrasonic probe.

10. The ultrasonic measurement apparatus according to claim 5,
wherein the target object image includes an image obtained by cutting the target object at a plane intersecting the axis of the target object and passing in the emission direction of the ultrasonic wave, and
wherein the needle image includes an image in which a tip of the puncture needle is viewed from an axial direction of the target object.

11. The ultrasonic measurement apparatus according to claim 5,
wherein the target object image includes an image obtained by cutting the target object at a plane passing in the emission direction of the ultrasonic wave along the axis of the target object, and
wherein the needle image includes an image in which the puncture needle is viewed from a direction intersecting an axial direction of the target object and intersecting the emission direction of the ultrasonic wave.
